(19) United States
(12) Patent Application Publication
(10) Pub. No.: US 2023/0089426 A1
(43) Pub. Date: Mar. 23, 2023

(54) METHODS FOR THE TREATMENT OF CANCER

(71) Applicant: Jounce Therapeutics, Inc., Cambridge, MA (US)

(72) Inventors: Yasmin RAMSAY, Cambridge, MA (US); Daniel Joseph FELITSKY, Cambridge, MA (US); Xiaoying XIAO, Cambridge, MA (US); Hugh M. COLE, Cambridge, MA (US)

(21) Appl. No.: 17/792,250

(22) PCT Filed: Jan. 13, 2021
(86) PCT No.: PCT/US2021/013266

§ 371 Date: Jul. 12, 2022

Related U.S. Application Data
(60) Provisional application No. 62/960,337, filed on Jan. 13, 2020.

(51) Int. Cl. C07K 16/28; C12Q 1/6886

(52) U.S. Cl. CPC C07K 16/2818; C12Q 1/6886; C12Q 2600/158; C12Q 2600/106; C07K 2317/75; C07K 2317/76

(57) ABSTRACT

The invention provides methods for the treatment of cancer. The invention also provides a method for identifying a subject whose cancer is likely to respond to combination ICOS agonist and PD1 antagonist therapy, the method comprising determining the RNA signature (Table 1) score of a sample of the cancer of the subject.

### SEQUENCE LISTING

The instant application contains a Sequence Listing which has been submitted electronically in ASCII format and is hereby incorporated by reference in its entirety. Said ASCII copy, created on Jan. 13, 2021, is named 51266-010WO2_Sequence_Listing_1_13_21_ST25 and is 42,430 bytes in size.

### FIELD OF THE INVENTION

The invention relates to methods of treating cancer and methods for selecting treatment approaches for cancer.

### BACKGROUND

ICOS (Inducible T-cell COStimulator; CD278) is a member of the B7/CD28/CTLA-4 immunoglobulin superfamily and is specifically expressed on T cells. Unlike CD28, which is constitutively expressed on T cells and provides co-stimulatory signals necessary for full activation of resting T cells, ICOS is expressed only after initial T cell activation.

ICOS has been implicated in diverse aspects of T cell responses (reviewed in Simpson et al., Curr. Opin. Immunol. 22: 326-332, 2010). It plays a role in the formation of germinal centers, T/B cell collaboration, and immunoglobulin class switching. ICOS-deficient mice show impaired germinal center formation and have decreased production of interleukin IL-10. These defects have been specifically linked to deficiencies in T follicular helper cells. ICOS also plays a role in the development and function of other T cell subsets, including Th1, Th2, and Th17. Notably, ICOS co-stimulates T cell proliferation and cytokine secretion associated with both Th1 and Th2 cells. Accordingly, ICOS knock-out mice demonstrate impaired development of autoimmune phenotypes in a variety of disease models, including diabetes (Th1), airway inflammation (Th2), and EAE neuro-inflammatory models (Th17).

In addition to its role in modulating T effector (Teff) cell function, ICOS also modulates T regulatory cells (Tregs). ICOS is expressed at high levels on Tregs, and has been implicated in Treg homeostasis and function.

Upon activation, ICOS, a disulfide-linked homodimer, induces a signal through the PI3K and AKT pathways. Subsequent signaling events result in expression of lineage specific transcription factors (e.g., T-bet, GATA-3) and, in turn, effects on T cell proliferation and survival.

ICOS ligand (ICOSL; B7-H2; B7RP1; CD275; GL50), also a member of the B7 superfamily, is the only ligand for ICOS and is expressed on the cell surfaces of B cells, macrophages, and dendritic cells. ICOSL functions as a non-covalently linked homodimer on the cell surface in its interaction with ICOS. Human ICOSL, although not mouse ICOSL, has been reported to bind to human CD28 and CTLA-4 (Yao et al., Immunity 34: 729-740, 2011).

### SUMMARY

The invention provides methods for treating a subject having cancer characterized by an elevated RNA signature score, the methods including administering to the subject an ICOS agonist and a PD1 antagonist.

The invention also provides methods for identifying a subject whose cancer is likely to respond to combination ICOS agonist and PD1 antagonist therapy, the methods including determining the RNA signature score of a sample of the cancer of the subject, wherein detection of an elevated RNA signature score in the sample indicates that the subject is likely to respond to the combination therapy.

The invention additionally provides methods of selecting a cancer therapy for a subject having cancer, the methods including determining the RNA signature score of a sample of the cancer of the subject, wherein detection of an elevated RNA signature score in the sample indicates selection of a combination of ICOS agonist and PD1 antagonist therapy for the subject.

The invention further provides methods of selecting a subject having cancer for combination ICOS agonist and PD1 antagonist therapy, the methods including determining the RNA signature score of a sample of the cancer of the subject, wherein detection of an elevated RNA signature score in the sample indicates selection of a subject for the combination therapy.

The invention also provides methods of determining whether a subject having cancer may develop an ICOShi CD4+ T cell population, the methods including determining the RNA signature score of a sample of the cancer of the subject, wherein detection of an elevated RNA signature score in the sample indicates that the subject may develop an ICOShi CD4+ T cell population.

In some embodiments, the methods further include administering an ICOS agonist and a PD-1 antagonist to the subject.

The invention also includes methods for increasing the length of duration of response to a PD1 antagonist in a subject having cancer, the methods including administering an ICOS agonist to the subject, wherein the cancer of the subject has an elevated RNA signature score.

In some embodiments, a subject having cancer with an elevated RNA signature score has improved tumor regression, duration of response, or RECIST criteria when treated with a combination of an ICOS agonist and a PD1 antagonist.

In some embodiments, the subject has improved overall response rate, progression free survival, stable disease, or overall survival.

In some embodiments, the subject has an increased level of ICOShi CD4+ T cells.

In some embodiments, the methods further include determining the RNA signature score of a sample of the cancer from the subject.

In some embodiments, the RNA signature score is determined by evaluation of RNA levels of the components of the RNA signature.

In some embodiments, the RNA signature score is determined by nanostring technology.

In some embodiments, determining of the RNA signature score includes detection of the levels of each RNA listed in Table 1, normalizing the levels of RNA listed in Table 1 against the levels of standards in Table 2, and weighting the normalized levels using the fourth column of Table 1.

In some embodiments, the RNA signature score is elevated if it is measured to be about 2.97 or above.

In some embodiments, the elevated RNA signature score is about 3.39 or above.

In some embodiments, the elevated RNA signature score is about 3.40 or above.

In some embodiments, the elevated RNA signature score is 3.40.

In some embodiments, the elevated RNA signature score is about 3.58 or above.

In some embodiments, the elevated RNA signature score is between about 2.97 and 3.58.

In some embodiments, the ICOS agonist is an antibody.

In some embodiments, the ICOS agonist antibody includes (a) a heavy chain including the amino acid sequence of SEQ ID NO: 1, or (b) a light chain including the amino acid sequence of SEQ ID NO: 2.

In some embodiments, the anti-ICOS antibody agonist includes (a) a heavy chain including the amino acid sequence of SEQ ID NO: 1, and (b) a light chain including the amino acid sequence of SEQ ID NO: 2.

In some embodiments, the ICOS agonist antibody is selected from the group consisting of JTX-2011, BMS-986226, and GSK3359609.

In some embodiments, the PD1 antagonist is directed against PD1.

In some embodiments, the PD1 antagonist is directed against PD-L1.

In some embodiments, the PD1 antagonist is an antibody.

In some embodiments, the PD1 antagonist antibody is selected from the group consisting of: JTX-4014, nivolumab, pidilizumab, lambrolizumab, pembrolizumab, cerniplimab, avelumab, atezolizumab tislelizumab, durvalumab, spartalizumab, genoliizurnab, BGB-A317, RG-7446, AMP-224, BMS-936559, AMP-514, MDX-1105, AB-011, RG7446/MPDL3280A, KD-033, AGEN-2034, STI-A1010, STI-A1110, TSR-042, CX-072, JNJ-63723283, and JNC-1.

In some embodiments, the PD1 antagonist antibody is JTX-4014.

In some embodiments, the cancer of the subject is selected from the group consisting of gastric cancer, breast cancer, which optionally is triple negative breast cancer (TNBC), non-small cell lung cancer (NSCLC), melanoma, renal cell carcinoma (RCC), bladder cancer, endometrial cancer, diffuse large B-cell lymphoma (DLBCL), Hodgkin's lymphoma, ovarian cancer, head and neck squamous cell cancer (HNSCC), anal cancer, biliary cancer, colorectal cancer, and esophageal cancer.

The invention also provides kits for use in determining whether to administer a combination of ICOS agonist and PD-1 antagonist therapy to a subject having cancer, the kits including primers and/or probes for detecting the components of an RNA signature as described herein in a sample of the cancer of the subject.

The invention further provides an ICOS agonist and a PD1 antagonist for use in the treatment of a subject having cancer characterized by an elevated RNA signature score.

In some embodiments of the ICOS agonist and PD1 antagonist for use, the subject having cancer characterized by an elevated RNA signature score has: (a) improved tumor regression, duration of response, or RECIST criteria when treated with a combination of an ICOS agonist and a PD1 antagonist; (b) improved overall response rate, progression free survival, stable disease, or overall survival; or (c) an increased level of ICOShi CD4+ T cells.

In some embodiments of the ICOS agonist and PD1 antagonist for use, the RNA signature score of a sample of the cancer from the subject is determined, wherein: (a) the RNA signature score is determined by evaluation of RNA levels of the components of the RNA signature; (b) the RNA signature score is determined by nanostring technology; or (c) determining of the RNA signature score comprises detection of the levels of each RNA listed in Table 1, normalizing the levels of RNA listed in Table 1 against the levels of standards in Table 2, and weighting the normalized levels using the fourth column of Table 1.

In some embodiments of the ICOS agonist and PD1 antagonist for use, (a) the RNA signature score is elevated if it is measured to be about 2.97 or above; (b) the elevated RNA signature score is about 3.39 or above; (c) the elevated RNA signature score is about 3.40 or above; (d) the elevated RNA signature score is 3.40; (e) the elevated RNA signature score is about 3.58 or above; or (f) the elevated RNA signature score is between about 2.97 and about 3.58.

In some embodiments of the ICOS agonist and PD1 antagonist for use, (a) the ICOS agonist is an antibody; (b) the ICOS agonist antibody comprises (i) a heavy chain comprising the amino acid sequence of SEQ ID NO: 1, or (ii) a light chain comprising the amino acid sequence of SEQ ID NO: 2; (c) the anti-ICOS antibody agonist comprises (i) a heavy chain comprising the amino acid sequence of SEQ ID NO: 1, and (ii) a light chain comprising the amino acid sequence of SEQ ID NO: 2; or (d) the ICOS agonist antibody is selected from the group consisting of JTX-2011, BMS-986226, and GSK3359609.

In some embodiments of the ICOS agonist and PD1 antagonist for use, (a) the PD1 antagonist is directed against PD1; (b) the PD1 antagonist is directed against PD-L1; (c) the PD1 antagonist is an antibody; (d) the PD1 antagonist antibody is selected from the group consisting of: JTX-4014, nivolumab, pidilizumab, lambrolizumab, pembrolizumab, cemiplirnab, avelumab, atezolizumab tislelizumab, durvalumab, spartalizumab, genolimzurnab, BGB-A317, RG-7446, AMP-224, BMS-936559, AMP-514, MDX-1105, AB-011, RG7446/MPDL3280A, KD-033, AGEN-2034, STI-A1010, STI-A1110, TSR-042, CX-072, JNJ-63723283, and JNC-1; or (e) the PD1 antagonist antibody is JTX-4014.

In some embodiments of the ICOS agonist and PD1 antagonist for use, the cancer of the subject is selected from the group consisting of gastric cancer, breast cancer, which optionally is triple negative breast cancer (TNBC), non-small cell lung cancer (NSCLC), melanoma, renal cell carcinoma (RCC), bladder cancer, endometrial cancer, diffuse large B-cell lymphoma (DLBCL), Hodgkin's lymphoma, ovarian cancer, head and neck squamous cell cancer (HNSCC), anal cancer, biliary cancer, colorectal cancer, and esophageal cancer.

The invention further provides an ICOS agonist for use in increasing the length of duration of response to a PD1 antagonist in a subject having cancer characterized by an elevated RNA signature score.

In some embodiments of the ICOS agonist for use, the subject having cancer characterized by an elevated RNA signature score has: (a) improved tumor regression, duration of response, or RECIST criteria when treated with a combination of an ICOS agonist and a PD1 antagonist; (b) improved overall response rate, progression free survival, stable disease, or overall survival; or (c) an increased level of ICOShi CD4+ T cells.

In some embodiments of the ICOS agonist for use, the RNA signature score of a sample of the cancer from the subject is determined, wherein: (a) the RNA signature score is determined by evaluation of RNA levels of the components of the RNA signature; (b) the RNA signature score is determined by nanostring technology; or (c) determining of the RNA signature score comprises detection of the levels of each RNA listed in Table 1, normalizing the levels of RNA listed in Table 1 against the levels of standards in Table 2, and weighting the normalized levels using the fourth column of Table 1.

In some embodiments of the ICOS agonist for use, (a) the RNA signature score is elevated if it is measured to be about 2.97 or above; (b) the elevated RNA signature score is about 3.39 or above; (c) the elevated RNA signature score is about 3.40 or above; (d) the elevated RNA signature score is 3.40; (e) the elevated RNA signature score is about 3.58 or above; or (f) the elevated RNA signature score is between about 2.97 and about 3.58.

In some embodiments of the ICOS agonist for use, (a) the ICOS agonist is an antibody; (b) the ICOS agonist antibody comprises (i) a heavy chain comprising the amino acid sequence of SEQ ID NO: 1, or (ii) a light chain comprising the amino acid sequence of SEQ ID NO: 2; (c) the anti-ICOS antibody agonist comprises (i) a heavy chain comprising the amino acid sequence of SEQ ID NO: 1, and (ii) a light chain comprising the amino acid sequence of SEQ ID NO: 2; or (d) the ICOS agonist antibody is selected from the group consisting of JTX-2011, BMS-986226, and GSK3359609.

In some embodiments of the ICOS agonist for use, (a) the PD1 antagonist is directed against PD1; (b) the PD1 antagonist is directed against PD-L1; (c) the PD1 antagonist is an antibody; (d) the PD1 antagonist antibody is selected from the group consisting of: JTX-4014, nivolumab, pidilizumab, lambrolizumab, pembrolizumab, cemiplimab, avelumab, atezolizumab tislelizumab, durvalumab, spartalizumab, genolimzurnab, BGB-A317, RG-7446, AMP-224, BMS-936559, AMP-514, MDX-1105, AB-011, RG7446/MPDL3280A, KD-033, AGEN-2034, STI-A1010, STI-A1110, TSR-042, CX-072, JNJ-63723283, and JNC-1; or (e) the PD1 antagonist antibody is JTX-4014.

In some embodiments of the ICOS agonist for use, the cancer of the subject is selected from the group consisting of gastric cancer, breast cancer, which optionally is triple negative breast cancer (TNBC), non-small cell lung cancer (NSCLC), melanoma, renal cell carcinoma (RCC), bladder cancer, endometrial cancer, diffuse large B-cell lymphoma (DLBCL), Hodgkin's lymphoma, ovarian cancer, head and neck squamous cell cancer (HNSCC), anal cancer, biliary cancer, colorectal cancer, and esophageal cancer.

Other features and advantages of the invention will be apparent from the following detailed description, the drawings, and the claims.

### BRIEF DESCRIPTION OF THE DRAWINGS

FIG. 1 shows RNA signature scores as predicting tumor response.

FIG. 2A shows RNA signature scores in the context of ICOS hi emergence. The mean values of 6.47 and 7.72 correspond to RNA signature scores as described elsewhere herein of 2.97 and 3.35, respectively.

FIG. 2B shows RNA signature scores in the context of subjects classified as responders/non-responders to treatment based on percent tumor reduction. The mean values of 7.00 and 8.27 correspond to RNA signature scores as described elsewhere herein of 3.13 and 3.51, respectively.

FIG. 3A shows RNA signature scores in the context of ICOS hi emergence in PD-1i naïve subjects. The mean values of 6.49 and 7.54 correspond to RNA signature scores as described elsewhere herein of 2.98 and 3.29, respectively.

FIG. 3B shows RNA signature scores in the context of subjects classified as responders/non-responders to treatment based on percent tumor reduction in PD-1i naïve subjects. The mean values of 6.81 and 8.42 correspond to RNA signature scores as described elsewhere herein of 3.07 and 3.56, respectively.

FIG. 4 shows tumor reductions for all prior anti-PD-1/anti-PD-L1 naïve subjects that had fresh pre-treatment tumor biopsies evaluated for the RNA signature score. The low, medium, and high RNA signature score cut-offs of 6.46, 7.85, and 8.5 correspond to RNA signature scores of as described elsewhere herein of 2.97, 3.39, and 3.58, respectively.

FIG. 5 shows clinical endpoints calculated for all prior anti-PD-1/anti-PD-L1 naïve subjects that had fresh pre-treatment tumor biopsies evaluated for the RNA signature score. The low, medium, and high RNA signature score cut-offs of 6.46, 7.85, and 8.5 correspond to RNA signature scores of 2.97, 3.39, and 3.58, respectively, as described elsewhere herein.

FIG. 6A shows tumor reductions and swimmers plots for subjects who had fresh pre-treatment tumor biopsies evaluated for the RNA signature score. The cut-off of 7.9 corresponds to an RNA signature score of 3.40 as described elsewhere herein.

FIG. 6B shows Kaplan Meier plots of clinical endpoints Progression Free Survival (PFS) and Overall Survival (OS), evaluated for subjects who had fresh pre-treatment tumor samples evaluated for the RNA signature score.

FIG. 7 is a Receiver Operating Characteristic (ROC) curve showing the relationship between the sensitivity and specificity of the RNA signature threshold. The cut-off of 7.914 corresponds to an RNA signature score of 3.40 as described elsewhere herein.

### DETAILED DESCRIPTION

Provided herein are methods of treating subjects having an elevated RNA signature score with combination ICOS agonist and PD-1 antagonist therapy. Also provided are methods of determining whether a subject will respond to combined ICOS agonist and PD-1 antagonist therapy and identifying such subjects. Additionally, compositions and kits for use in carrying out the methods described herein are provided. These and other methods, compositions, and kits of the invention are described further as follows.

These inventions are based, in part, on the observation that subjects having an elevated RNA signature score, as calculated by the methods described herein, were more likely to:
Respond (i.e., have an objective response) to combination ICOS agonist and PD-1 antagonist therapy,
Experience clinical benefit as a result of the combination therapy,
Exhibit the presence of an ICOShi population of CD4+ T-cells,
Experience a Progression Free Survival (PFS) of at least 6 months as a result of the combination therapy, and
Experience a longer period of overall survival as a result of the combination therapy.

Furthermore, the data suggests that the elevated RNA signature was more predictive of response than biomarkers previously associated with response to PD-1 antagonist therapy (e.g., a PD-L1 IHC biomarker, which was not found to correlate with response in the combination therapy clinical studies reported herein), suggesting the utility of the RNA signature in predicting a response to the combination therapy distinct from the prediction of response to PD-1 antagonist therapy alone. Further in support of this hypothesis, it is notable that PD-1 antagonist therapy has not been associated with the emergence of ICOShi CD4+ T cells.

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety. The Accession numbers (see Tables 1 and 2) refer to the respective sequences available by the accession numbers on Jan. 13, 2020.

### I. Definitions

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that embodiments of the invention described herein include “consisting” and/or “consisting essentially of” embodiments.

As used herein, the singular form “a,” “an,” and “the” includes plural references unless indicated otherwise.

Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

As is understood by one skilled in the art, reference to “about” a value or parameter herein includes (and describes) embodiments that are directed to that value or parameter per se. For example, description referring to “about X” includes description of “X.” In the context of RNA signature score values, the term “about” includes the stated score and scores within 0.1% thereof.

The terms “nucleic acid molecule,” “nucleic acid,” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown below. Amino acid substitutions may be introduced into an antibody of interest and the products screened for a desired activity, for example, retained/improved antigen binding, decreased immunogenicity, or improved ADCC or CDC.

| Original Residue | Exemplary Substitutions |
|---|---|
| Ala (A) | Val; Leu; Ile |
| Arg (R) | Lys; Gin; Asn |
| Asn (N) | Gin; His; Asp, Lys; Arg |
| Asp (D) | Glu; Asn |
| Cys (C) | Ser; Ala |
| Gin (Q) | Asn; Glu |
| Glu (E) | Asp; Gin |
| Gly (G) | Ala |
| His (H) | Asn; Gin; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gin; Asn |
| Met (M) | Leu; Phe; He |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, lie;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

“ICOS” and “inducible T-cell costimulatory” as used herein refer to any native ICOS that results from expression and processing of ICOS in a cell. The term includes ICOS from any vertebrate source, including mammals such as primates (e.g., humans and cynomolgus monkeys) and rodents (e.g., mice and rats), unless otherwise indicated. The term also includes naturally occurring variants of ICOS, e.g., splice variants or allelic variants. The amino acid sequence of an exemplary human ICOS precursor protein, with signal sequence (amino acids 1-20) is shown in SEQ ID NO: 11. The amino acid sequence of an exemplary mature human ICOS is shown in SEQ ID NO: 12. The intracellular portion of ICOS is indicated in Table 4 by underlining within SEQ ID NOs: 11 and 12. The amino acid sequence of an exemplary mouse ICOS precursor protein, with signal sequence (amino acids 1-20) is shown in SEQ ID NO: 13. The amino acid sequence of an exemplary mature mouse ICOS is shown in SEQ ID NO: 14. The amino acid sequence of an exemplary rat ICOS precursor protein, with signal sequence (amino acids 1-20) is shown in SEQ ID NO: 15. The amino acid sequence of an exemplary mature rat ICOS is shown in SEQ ID NO: 16. The amino acid sequence of an exemplary cynomolgus monkey ICOS precursor protein, with signal sequence (amino acids 1-20) is shown in SEQ ID NO: 17. The amino acid sequence of an exemplary mature cynomolgus monkey ICOS is shown in SEQ ID NO: 18.

The term “specifically binds” to an antigen or epitope is a term that is well-understood in the art, and methods to determine such specific binding are also well known in the art. A molecule is said to exhibit “specific binding” or “preferential binding” if it reacts or associates more frequently, more rapidly, with greater duration, and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an ICOS epitope is an antibody that binds this epitope with greater affinity, avidity, more readily, and/or with greater duration than it binds to other ICOS epitopes or non-ICOS epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, “substantially pure” refers to material which is at least 50% pure (that is, free from contaminants), more preferably, at least 90% pure, more preferably, at least 95% pure, yet more preferably, at least 98% pure, and most preferably, at least 99% pure.

As used herein, the term “epitope” refers to a site on a target molecule (for example, an antigen, such as a protein, nucleic acid, carbohydrate, or lipid) to which an antigen-binding molecule (for example, an antibody, antibody fragment, or scaffold protein containing antibody binding regions) binds. Epitopes often include a chemically active surface grouping of molecules such as amino acids, polypeptides, or sugar side chains and have specific three-dimensional structural characteristics as well as specific charge characteristics. Epitopes can be formed both from contiguous and/or juxtaposed noncontiguous residues (for example, amino acids, nucleotides, sugars, or lipid moieties) of the target molecule. Epitopes formed from contiguous residues, also called linear epitopes (for example, amino acids, nucleotides, sugars, or lipid moieties), typically are retained on exposure to denaturing solvents whereas epitopes formed from non-contiguous residues, also called non-linear or conformational epitopes, are formed by tertiary folding, and typically are lost on treatment with denaturing solvents. An epitope may include, but is not limited to, at least 3, at least 5, or 8-10 residues (for example, amino acids or nucleotides). In some examples, an epitope is less than 20 residues (for example, amino acids or nucleotides) in length, less than 15 residues, or less than 12 residues.

Two antibodies may bind to the same epitope within an antigen, or to overlapping epitopes, if they exhibit competitive binding for the antigen. Accordingly, in some embodiments, an antibody is said to “cross-compete” with another antibody if it specifically interferes with the binding of the antibody to the same or an overlapping epitope.

The term “antibody” herein is used in the broadest sense and encompasses various antibody structures, including but not limited to, monoclonal antibodies, polyclonal antibodies, multispecific antibodies (for example, bispecific (such as Bi-specific T-cell engagers) and trispecific antibodies), and antibody fragments as long as they exhibit a desired antigen-binding activity.

The term antibody includes, but is not limited to, fragments that are capable of binding to an antigen, such as Fv, single-chain Fv (scFv), Fab, Fab′, di-scFv, sdAb (single domain antibody), and (Fab′)2 (including a chemically linked F(ab′)2). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc. Furthermore, for all antibody constructs provided herein, variants having the sequences from other organisms are also contemplated. Thus, if a human version of an antibody is disclosed, one of skill in the art will appreciate how to transform the human sequence-based antibody into a mouse, rat, cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example. The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, and/or a combination of the Kabat, Chothia, AbM, and/or contact definitions. Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991)). The AbM definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, H26-H35B of H1, 50-58 of H2, and 95-102 of H3. The Contact definition can include, for example, CDRs (CDR-L1, CDR-L2, CDR-L3, CDR-H1, CDR-H2, and CDR-H3) at amino acid residues 30-36 of L1, 46-55 of L2, 89-96 of L3, 30-35 of H1, 47-58 of H2, and 93-101 of H3. The Chothia definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, 26-32 . . . 34 of H1, 52-56 of H2, and 95-102 of H3. With the exception of CDR1 in VH, CDRs generally comprise the amino acid residues that form the hypervariable loops. The various CDRs within an antibody can be designated by their appropriate number and chain type, including, without limitation as: a) CDR-L1, CDR-L2, CDR-L3, CDR-H1, CDR-H2, and CDR-H3; b) CDRL1, CDRL2, CDRL3, CDRH1, CDRH2, and CDRH3; c) LCDR-1, LCDR-2, LCDR-3, HCDR-1, HCDR-2, and HCDR-3; or d) LCDR1, LCDR2, LCDR3, HCDR1, HCDR2, and HCDR3; etc. The term “CDR” is used herein to also encompass HVR or a “hyper variable region,” including hypervariable loops. Exemplary hypervariable loops occur at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)).

The term “heavy chain variable region” as used herein refers to a region comprising at least three heavy chain CDRs. In some embodiments, the heavy chain variable region includes the three CDRs and at least FR2 and FR3. In some embodiments, the heavy chain variable region includes at least heavy chain HCDR1, framework (FR) 2, HCDR2, FR3, and HCDR3. In some embodiments, a heavy chain variable region also comprises at least a portion of an FR1 and/or at least a portion of an FR4.

The term “heavy chain constant region” as used herein refers to a region comprising at least three heavy chain constant domains, CH1, CH2, and CH3. Of course, non-function-altering deletions and alterations within the domains are encompassed within the scope of the term “heavy chain constant region,” unless designated otherwise. Non-limiting exemplary heavy chain constant regions include γ, δ, and α. Non-limiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising at least three light chain CDRs. In some embodiments, the light chain variable region includes the three CDRs and at least FR2 and FR3. In some embodiments, the light chain variable region includes at least light chain LCR1, framework (FR) 2, LCD2, FR3, and LCD3. For example, a light chain variable region may comprise light chain CDR1, framework (FR) 2, CDR2, FR3, and CDR3. In some embodiments, a light chain variable region also comprises at least a portion of an FR1 and/or at least a portion of an FR4.

The term “light chain constant region” as used herein refers to a region comprising a light chain constant domain, C_{L}. Non-limiting exemplary light chain constant regions include A and K. Of course, non-function-altering deletions and alterations within the domains are encompassed within the scope of the term “light chain constant region,” unless designated otherwise.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

An “acceptor human framework” for the purposes herein is a framework comprising the amino acid sequence of a light chain variable domain (V_{L}) framework or a heavy chain variable domain (V_{H}) framework derived from a human immunoglobulin framework or a human consensus framework, as defined below. An acceptor human framework derived from a human immunoglobulin framework or a human consensus framework can comprise the same amino acid sequence thereof, or it can contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the V_{L} acceptor human framework is identical in sequence to the V_{L} human immunoglobulin framework sequence or human consensus framework sequence.

“Affinity” refers to the strength of the sum total of noncovalent interactions between a single binding site of a molecule (for example, an antibody) and its binding partner (for example, an antigen). The affinity of a molecule X for its partner Y can generally be represented by the dissociation constant (KD). Affinity can be measured by common methods known in the art (such as, for example, ELISA KD, KinExA, bio-layer interferometry (BLI), and/or surface plasmon resonance devices (such as a BIAcore® device), including those described herein).

The term “KD,” “Kd,” “Kd,” or “Kd value” as used herein, refers to the equilibrium dissociation constant of an antibody-antigen interaction.

The term “biological activity” refers to any one or more biological properties of a molecule (whether present naturally as found in vivo, or provided or enabled by recombinant means). Biological properties include, but are not limited to, binding a receptor, inducing cell proliferation, inhibiting cell growth, inducing other cytokines, inducing apoptosis, and enzymatic activity. In some embodiments, biological activity of an ICOS protein includes, for example, costimulation of T cell proliferation and cytokine secretion associated with Th1 and Th2 cells; modulation of Treg cells; effects on T cell differentiation including modulation of transcription factor gene expression; induction of signaling through PI3K and AKT pathways; and mediating ADCC.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

The phrase “substantially different,” as used herein, denotes a sufficiently high degree of difference between two numeric values such that one of skill in the art would consider the difference between the two values to be of statistical significance within the context of the biological characteristic measured by said values. In some embodiments, the two substantially different numeric values differ by greater than about any one of 10%, 20%, 25%, 30%, 35%, 40%, 45%, 50%, 60%, 70%, 80%, 90%, or 100%.

The phrase “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 20%, 25%, 30%, 35%, 40%, 45%, 50%, 60%, 70%, 80%, 90%, or 100% compared to the reference value.

The phrase “substantially increased,” as used herein, denotes a sufficiently high degree of increase between a numeric value and a reference numeric value such that one of skill in the art would consider the difference between the two values to be of statistical significance within the context of the biological characteristic measured by said values. In some embodiments, the substantially increased numeric values is increased by greater than about any one of 10%, 20%, 25%, 30%, 35%, 40%, 45%, 50%, 60%, 70%, 80%, 90%, or 100% compared to the reference value.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated.”

The terms “individual,” “patient,” or “subject” are used interchangeably herein to refer to an animal, for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

The term “sample” or “patient sample” as used herein, refers to a composition that is obtained or derived from a subject of interest that contains a cellular and/or other molecular entity that is to be characterized and/or identified, for example, based on physical, biochemical, chemical, and/or physiological characteristics. For example, the phrase “test sample,” and variations thereof, refers to any sample obtained from a subject of interest that would be expected or is known to contain a cellular and/or molecular entity that is to be characterized. By “tissue or cell sample” is meant a collection of similar cells obtained from a tissue of a subject or patient. The source of the tissue or cell sample may be blood (e.g., peripheral blood) or any blood constituents; solid tissue as from a fresh, frozen, and/or preserved organ or tissue sample or biopsy or aspirate; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like. In some embodiments, a sample includes peripheral blood obtained from a subject or patient, which includes CD4+ cells. In some embodiments, a sample includes CD4+ cells isolated from peripheral blood. In some embodiments, a sample is a sample of peripheral blood mononuclear cells (PBMCs).

A “control,” “control sample,” “reference,” or “reference sample” as used herein, refers to any sample, standard, or level that is used for comparison purposes. A control or reference may be obtained from a healthy and/or non-diseased sample. In some examples, a control or reference may be obtained from an untreated sample or patient. In some examples, a reference is obtained from a non-diseased or non-treated sample of a subject individual. In some examples, a reference is obtained from one or more healthy individuals who are not the subject or patient. In some embodiments, a control sample, reference sample, reference cell, or reference tissue is obtained from the patient or subject at a time point prior to one or more administrations of a treatment (e.g., one or more anti-cancer treatments), or prior to being subjected to any of the methods of the invention.

A “disease” or “disorder” as used herein refers to a condition where treatment is needed and/or desired. In some embodiments, the disease or disorder is cancer.

“Cancer” and “tumor,” as used herein, are interchangeable terms that refer to any abnormal cell or tissue growth or proliferation in an animal. As used herein, the terms “cancer” and “tumor” encompass solid and hematological/lymphatic cancers and also encompass malignant, pre-malignant, and benign growth, such as dysplasia. Examples of cancer include but are not limited to, carcinoma, lymphoma, blastoma, sarcoma, and leukemia. More particular non-limiting examples of such cancers include gastric cancer, breast cancer (e.g., triple negative breast cancer (TNBC)), non-small cell lung cancer (NSCLC), squamous cell cancer, small-cell lung cancer, pituitary cancer, esophageal cancer, astrocytoma, soft tissue sarcoma, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, colon cancer, colorectal cancer, endometrial or uterine carcinoma (including uterine corpus endometrial carcinoma), salivary gland carcinoma, kidney cancer, renal cancer, liver cancer, prostate cancer, vulval cancer, thyroid cancer, hepatic carcinoma, brain cancer, testis cancer, cholangiocarcinoma, gallbladder carcinoma, melanoma, and various types of head and neck cancer. These cancers, and others, can be treated or analyzed according to the methods of the invention.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example, metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering an anti-cancer therapy. “Ameliorating” also includes shortening or reduction in duration of a symptom.

In the context of cancer, the term “treating” includes any or all of: inhibiting growth of cancer cells, inhibiting replication of cancer cells, lessening of overall tumor burden, and ameliorating one or more symptoms associated with the disease.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce,” “inhibit,” or “prevent” do not denote or require complete prevention over all time.

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (for example, severity of disease, progression/non-progression/improvement, etc.). While the present disclosure may provide exemplary predetermined levels, it is well-known that cutoff values may vary depending on the nature of the immunoassay (for example, antibodies employed, etc.). It further is well within the skill of one of ordinary skill in the art to adapt the disclosure herein for other immunoassays to obtain immunoassay-specific cutoff values for those other immunoassays based on this disclosure. Whereas the precise value of the predetermined cutoff/level may vary between assays, correlations as described herein (if any) may be generally applicable.

An “RNA signature score” as described herein is calculated by determining RNA levels for each gene of the gene signature of Table 1 and a normalization gene set (see, e.g., Table 2; first 10 genes, or all 11 genes). In some embodiments, the RNA levels are log 2 transformed. The arithmetic mean of the log (2) transformed RNA levels of the normalization genes is obtained, and this number is subtracted from the log (2) transformed RNA levels for each of the signature genes, and this value is added to 10. This gives the housekeeper normalized, log 2 transformed value for each signature gene. Next, these values are transformed by using them as exponents (taking 2 to the power of each value) followed by a log 10 transformation to give the unweighted, log 10 transformed housekeeper normalized expression levels. Next, weighting of each signature gene is done by multiplying unweighted, log 10 transformed housekeeper normalized expression levels against the respective factor indicated for each gene in the fourth column of Table 1. A final weighted score is then obtained by adding the weighted numbers for each gene of the signature. In some embodiments, a gene signature is considered to be “elevated” if it is about 2.97 or above. In some embodiments, a gene signature is considered to be “elevated” if it is about 3.39 or above. In some embodiments, a gene signature is considered to be “elevated” if it is 3.40 or is about 3.40 or above. In some embodiments, a gene signature is considered to be “elevated” if it is about 3.58 or above. In some embodiments, a gene signature is considered to be “elevated” if it is about 2.97 to 3.58 or above.

In some embodiments, the terms “elevated levels of ICOS,” “elevated ICOS levels,” “ICOS at an elevated level,” “ICOS^{HIGH},” and “ICOS^{hi}” refer to increased levels of ICOS in cells (e.g., CD4+ T cells) of a subject, e.g., in a peripheral blood sample of the subject, after treatment of the subject with one or more anti-cancer therapies. The increased levels can be determined relative to a control which may be, e.g., a peripheral blood sample from the subject being treated, but either before any treatment with the one or more anti-cancer therapies at all, or before treatment with a second or further cycle of the one or more anti-cancer therapies. Alternatively, the control can be a level from a matched sample (e.g., a peripheral blood sample) of a healthy individual. In some embodiments, the level of ICOS is determined at the level of expressed protein, which may be detected in some embodiments using an antibody directed to an intracellular portion of ICOS. In some embodiments, the detection using such an antibody is done by use of flow cytometry. In some embodiments, an increase of at least 2-fold (e.g., at least 3-fold, 4-fold, 5-fold, 7.5-fold, 10-fold, or 15-fold) in mean fluorescence intensity (MFI), relative to a control, indicates detection of elevated ICOS levels. In some embodiments, detection of an increase in ICOS levels in at least 5% (e.g., at least 10%, 20%, 30%, 40%, 50%, 60%, 70%, 80%, or 90%) of CD4+ T cells in a peripheral blood sample indicates a subject having an ICOS hi sample. In some embodiments, an increase of at least 2-fold (e.g., at least 3-fold, 4-fold, 5-fold, 7.5-fold, 10-fold, or 15-fold) in mean fluorescence intensity (MFI), relative to a control, in at least 5% (e.g., at least 10%, 20%, 30%, 40%, 50%, 60%, 70%, 80%, or 90%) of CD4+ T cells in a peripheral blood sample indicates detection of elevated ICOS levels. In some embodiments, elevated ICOS levels refer to an increase in total ICOS expression levels (e.g., mRNA levels or protein levels) in CD4+ T cells in the peripheral blood test sample of about 10%, 20%, 30%, 40%, 50%, 60%, 70%, 80%, 85%, 90%, 95%, 100%, or greater relative to a control sample. In some embodiments, elevated ICOS levels refers to an increase in total ICOS expression levels (e.g., mRNA levels or protein levels) in the CD4+ T cells in a peripheral blood sample of about at least 1.1×, 2×, 3×, 4×, 5×, 10×, 15×, 20×, 30×, 40×, 50×, 100×, 500×, 1000×, or greater relative to a control sample.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce, or arrest an activity, function, and/or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time.

As used herein, “delaying development of a disease” means to defer, hinder, slow, retard, stabilize, suppress, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

A “therapeutically effective amount” of a substance/molecule, agonist, or antagonist may vary according to factors such as the disease state, age, sex, and weight of the individual, and the ability of the substance/molecule, agonist, or antagonist to elicit a desired response in the individual. A therapeutically effective amount is also one in which any toxic or detrimental effects of the substance/molecule, agonist, or antagonist are outweighed by the therapeutically beneficial effects. A therapeutically effective amount may be delivered in one or more administrations. A therapeutically effective amount refers to an amount effective, at dosages, and for periods of time necessary, to achieve the desired therapeutic and/or prophylactic result. The therapeutically effective amount of the treatment of the invention can be measured by various endpoints commonly used in evaluating cancer treatments, including, but not limited to: extending survival (including OS and PFS); resulting in an objective response (including a CR or a PR); tumor regression, tumor weight or size shrinkage, longer time to disease progression, increased duration of survival, longer PFS, improved OS rate, increased duration of response, and improved quality of life and/or improving signs or symptoms of cancer.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time, or where the administration of one therapeutic agent falls within a short period of time (e.g., within one day) relative to administration of the other therapeutic agent. For example, the two or more therapeutic agents are administered with a time separation of no more than about a specified number of minutes.

The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes.

As used herein, “in conjunction with” refers to administration of one treatment modality in addition to another treatment modality. As such, “in conjunction with” refers to administration of one treatment modality before, during, or after administration of the other treatment modality to the individual.

The terms “label” and “detectable label” mean a moiety attached to a polynucleotide or polypeptide to render a reaction (for example, polynucleotide amplification or antibody binding) detectable. The polynucleotide or polypeptide comprising the label may be referred to as “detectably labeled.” Thus, the term “labeled binding protein” refers to a protein with a label incorporated that provides for the identification of the binding protein. The term “labeled oligonucleotide,” “labeled primer,” “labeled probe,” etc. refers to a polynucleotide with a label incorporated that provides for the identification of nucleic acids that comprise or are hybridized to the labeled oligonucleotide, primer, or probe. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In some embodiments, the moiety itself may not be detectably labeled but may become detectable upon reaction with yet another moiety.

The term “conjugate” refers to an antibody that is chemically linked to a second chemical moiety, such as a therapeutic or cytotoxic agent. The term “agent” includes a chemical compound, a mixture of chemical compounds, a biological macromolecule, or an extract made from biological materials. In some embodiments, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, the conjugate antibody may be a detectably labeled antibody used as the detection antibody.

As used herein, the term “flow cytometry” generally refers to a technique for characterizing biological particles, such as whole cells or cellular constituents, by flow cytometry. Methods for performing flow cytometry on samples of immune cells are well known in the art (see e.g., Jaroszeski et al., Method in Molecular Biology (1998), vol. 91: Flow Cytometry Protocols, Humana Press; Longobanti Givan, (1992) Flow Cytometry, First Principles, Wiley Liss). All known forms of flow cytometry are intended to be included, particularly fluorescence activated cell sorting (FACS), in which fluorescent labeled molecules are evaluated by flow cytometry.

The term “amplification” refers to the process of producing one or more copies of a nucleic acid sequence or its complement. Amplification may be linear or exponential (e.g., PCR).

The technique of “polymerase chain reaction” or “PCR” as used herein generally refers to a procedure wherein a specific region of nucleic acid, such as RNA and/or DNA, is amplified as described, for example, in U.S. Pat. No. 4,683,195. Generally, oligonucleotide primers are designed to hybridize to opposite strands of the template to be amplified, a desired distance apart. PCR can be used to amplify specific RNA sequences, specific DNA sequences from total genomic DNA, and cDNA transcribed from total cellular RNA, bacteriophage or plasmid sequences, etc.

“Quantitative real time PCR” or “qRT-PCR” refers to a form of PCR wherein the PCR is performed such that the amounts, or relative amounts of the amplified product can be quantified. This technique has been described in various publications including Cronin et al., Am. J. Pathol. 164(1):35-42 (2004); and Ma et al., Cancer Cell 5:607-616 (2004).

The term “target sequence,” “target nucleic acid,” or “target nucleic acid sequence” refers generally to a polynucleotide sequence of interest, e.g., a polynucleotide sequence that is targeted for amplification using, for example, qRT-PCR.

The term “detection” includes any means of detecting, including direct and indirect detection.

### II. Determination of RNA Signatures

In some embodiments, the methods of the invention include determining the RNA signature score of a sample of a cancer of a subject. Accordingly, an RNA signature score can be determined in a sample of tissue including cancer (e.g., a tumor sample, a blood sample, or a tissue sample wherein the tissue comprises cancer cells) that is obtained from a subject.

a. Sample Preparation

Cancers that can be analyzed according to the present invention can optionally be primary, metastatic, or recurrent, and may be of any type (e.g., as listed elsewhere herein). Furthermore, the cancers may be of any stage including, e.g., Stage I, II, III, or IV, and/or of any histology. A subject having the cancer can be of any age or gender, and may have any treatment history and/or extent or duration of disease or remission.

The cancer sample can be obtained using a variety of different procedures that are selected based on factors including, for example, the type, location, and size of the cancer. Exemplary methods include tissue biopsy, e.g., needle biopsy (e.g., fine needle aspiration, core needle biopsy, and image-guided biopsy); surgical biopsy (e.g., incisional biopsy or excisional biopsy); liquid biopsy (e.g., by obtaining circulating tumor cells); endoscopic biopsy; and scrape or brush biopsy.

Samples are processed for detection of RNA signatures using standard methods, which are selected based on, e.g., the type of cancer and the assay format to be used. In certain embodiments, the tumor tissue can be microdissected from the remaining sample prior to isolation of RNA. For example, the samples can be fixed using, e.g., neutral buffered formalin, glutaraldehyde, or paraformaldehyde. In some examples, a tissue sample fixed in formalin is also embedded in paraffin to prepare a formalin-fixed paraffin-embedded (or FFPE) tissue sample. The tissue sample can be sectioned and assayed as a fresh specimen. Alternatively, the tissue sample can be frozen for further processing, e.g., sectioning or nucleic acid extraction. In other examples, the samples can be in the form of a tissue or cell extract, or can be in the form of isolated, individual cells. Accordingly, the sample can be a solid tissue sample or slice, fluid derived from a tumor, fraction, or extract, lymph tissue, blood, or other tissue comprising the cancer cells. Moreover, the cancer cells can be cultured, washed, or otherwise selected to remove non-cancerous cells from the sample, and optionally the cancer cells can be sorted by fluorescence activated cell sorting or other cell sorting technique. Detection can be carried out on tissues, cells, tissue extracts, cell extracts, whole cell lysates, protein extracts, and nucleic acid extracts (e.g., RNA extracts). With respect to the latter, RNA can be extracted from cells using standard RNA extraction techniques and kits including, for example, acid guanidinium-acid-phenol extraction (TRIzol and TRI reagent), phenol/guanidine isothiocyanate extraction (RNAzoIB Biogenesis), silica technology and glass fiber filters (e.g. RNeasy RNA preparation kits (Qiagen)), magnetic bead technology (e.g., dynabeads mRNA DIRECT micro), lithium chloride and urea isolation, oligo(dt)-cellulose column chromatography, and non-column poly (A)+ purification.

b. Detection Methods

The components of the RNA signature can be detected in samples by analysis of transcribed polynucleotides, variants, portions, or reverse transcripts thereof (e.g., pre-mRNA, mRNA, splice variants, or cDNA; see, e.g., the exemplary target regions noted in Table 1). RNA detection methods that can be used include, e.g., nucleic acid sequence based amplification (NASBA) combined with molecular beacon detection molecules (Compton, Nature 350(6313):91-92, 1991), a flap endonuclease-based assay (e.g., Invader™, Third Wave Technologies), direct mRNA capture with branched DNA (QuanitGene™ Panomics) or Hybrid Capture™ (Digene), RNA-seq, RT-PCR, quantitative PCR, microarray analysis, ligase chain reaction, RNAse protection, nuclease protection combined with array detection (e.g., ArrayPlate™, HTG Molecular, Tucson, Ariz.; Martel et al., Assay and Drug Dev. Tech. 1(1):61-72, 2002), northern blotting, and nuclear run-on assays. Other approaches include hybridization-based methods employing a capture probe and a reporter probe, wherein the capture probe includes a sequence coupled to an immobilization tag for immobilization of a complex including the capture probe, analyte, and detection probe for analysis (e.g., a NanoString® system, such as the nCounter® Analysis System; NanoString® Technologies, Seattle, Wash.). Alternatively, RNA can be analyzed by hybridization of tissue samples with labeled probes. Expression of the biomarkers can also be analyzed at the protein level using, e.g., immunological based methods (e.g., immunohistochemistry (IHC), ELISA, FACS, capillary electrophoresis, HPLC, TLC, RIA, Western blotting, immunofluorescence, and proteomic methods (e.g., mass spectrometry). Additional details concerning exemplary methods that can be used to detect RNA are provided below.

In some embodiments, the methods provided herein include measuring an RNA (e.g., mRNA) level. In some embodiments, the methods provided herein include measuring an RNA signature, e.g., a plurality of RNA levels that are predictive of or correlated to improved responses to combined ICOS agonist and PD-1 antagonist therapy as described herein. In some embodiments, the RNA signature includes at least two, at least three, at least four, at least five, at least six, at least seven, at least eight, at least nine, at least ten, at least eleven, at least twelve, at least thirteen, at least fourteen, at least fifteen, at least sixteen, at least seventeen, or at least eighteen RNA levels, the RNA levels being levels of RNAs selected from Table 1.

Any suitable method of determining RNA (e.g., mRNA) levels may be used. Methods for the evaluation of RNA include, for example, hybridization assays using complementary nucleic acid probes (such as in situ hybridization using labeled riboprobes specific for target sequences, Northern blot, and related techniques), various nucleic acid amplification assays (such as RT-PCR using complementary primers specific for target sequences and other amplification type detection methods, such as, for example, branched DNA, SISB A, TMA and the like), and sequencing-based assays (e.g., RNA-seq).

Accordingly, in some embodiments, the RNA (e.g., mRNA) level is determined by the use of Nanostring technologies.

In some embodiments, the RNA (e.g., mRNA) level is determined by quantitative RT-PCR. In some embodiments, the mRNA level is determined by digital PCR. In some embodiments, the mRNA level is determined by RNA-Seq. In some embodiments, the mRNA level is determined by RNase Protection Assay (RPA). In some embodiments, the mRNA level is determined by Northern blot. In some embodiments, the mRNA level is determined by in situ hybridization (ISH). In some embodiments, the mRNA level is determined by a method selected from quantitative RT-PCR, microarray, digital PCR, RNA-Seq, RNase Protection Assay (RPA), Northern blot, and in situ hybridization (ISH).

RNA-seq is a technique based on enumeration of RNA transcripts using next-generation sequencing methodologies. The level of an mRNA is determined using the frequency of observation of fragments of its sequence (see, Wang et al., Nat. Rev. Genet. 10:57-63, 2009).

Northern blotting involves the use of electrophoresis to separate RNA samples by size, and detection with hybridization probes complementary to part of or the entire target sequence (see, e.g., Trayhurn, Northern Blotting. Pro. Nutrition Soc. 55:583-589, 1996).

Quantitative RT-PCR involves reverse-transcribing mRNA and then amplifying the resulting cDNA by polymerase chain reaction (PCR), which can be monitored in real time, e.g., by measuring fluorescence, wherein dye signal is a readout of the amount of product. The dye can be, e.g., an intercalating dye, or a dye attached to a probe also including a quencher, wherein degradation of the probe releases the dye and results in fluorescence, the degradation being catalyzed by an exonuclease activity driven by product formation, as in the TaqMan® assay. In some embodiments, a method for detecting a target mRNA in a biological sample includes producing cDNA from the sample by reverse transcription using at least one primer; amplifying the resulting cDNA using a target polynucleotide as sense and antisense primers to amplify target cDNAs therein; and detecting the presence of the amplified target cDNA. In addition, such methods can include one or more steps that allow one to determine the levels of target mRNA in a biological sample (e.g., by simultaneously examining the levels a reference mRNA sequence, e.g., a “housekeeping” gene such as the reference RNAs described herein). Optionally, the sequence of the amplified target cDNA can be determined.

In digital PCR, a sample is partitioned into a plurality of reaction areas and PCR is conducted in the areas. The number of areas that are positive, i.e., in which detectable product formation occurs, can be used to determine the level of the target sequence in the original sample.

In an RPA, a sample is contacted with a probe under hybridization conditions and then with a single-stranded RNA nuclease. Formation of double-stranded complexes of probe with target protect the probe from degradation, such that the amount of probe remaining can be used to determine the level of the target.

In ISH, a cell or tissue sample is contacted with a probe that hybridizes to a target RNA and hybridization is detected to determine the level of the target.

In some embodiments, the methods include protocols in which mRNAs, such as target mRNAs, are detected in a tissue or cell sample, or RNA extracted therefrom, using microarrays. In these assays, test and control mRNA samples from test and control tissue or cell samples are reverse transcribed and labeled to generate cDNA probes. The probes are then hybridized to an array of nucleic acids immobilized on a solid support. The array is configured such that the sequence and position of each member of the array is known. Hybridization of a labeled probe with a particular array member indicates that the sample from which the probe was derived expresses that gene. Microarray technology utilizes nucleic acid hybridization techniques and computing technology to evaluate the mRNA expression profile of thousands of genes within a single experiment (see, e.g., WO 01/75166; U.S. Pat. Nos. 5,700,637; 5,445,934; 5,807,522; Lockart, Nature Biotechnology 14:1675-1680, 1996; Cheung et al., Nature Genetics 21(Suppl):15-19, 1999). DNA microarrays are miniature arrays containing gene fragments that are either synthesized directly onto or spotted onto glass or other substrates. Thousands of genes are usually represented in a single array. A typical microarray experiment can involve the following steps: 1) preparation of fluorescently labeled target from RNA isolated from the sample, 2) hybridization of the labeled target to the microarray, 3) washing, staining, and scanning of the array, 4) analysis of the scanned image, and 5) generation of gene expression profiles. Two types of DNA microarrays are oligonucleotide (usually 25 to 70 mers) arrays and gene expression arrays containing PCR products prepared from cDNAs. In forming an array, oligonucleotides can be either prefabricated and spotted to the surface or directly synthesized onto the surface (in situ). In some embodiments, a DNA microarray is a single-nucleotide polymorphism (SNP) microarrays, e.g., Affymetrix® SNP Array 6.0.

The Affymetrix GeneChip® system is a commercially available microarray system that comprises arrays fabricated by direct synthesis of oligonucleotides on a glass surface. In probe/gene arrays, oligonucleotides, usually 25 mers, are directly synthesized onto a glass wafer by a combination of semiconductor-based photolithography and solid phase chemical synthesis technologies. Each array contains up to 400,000 different oligos and each oligo is present in millions of copies. Since oligonucleotide probes are synthesized in known locations on the array, the hybridization patterns and signal intensities can be interpreted in terms of gene identity and relative levels by the Affymetrix Microarray Suite software. Each gene is represented on the array by a series of different oligonucleotide probes. Each probe pair consists of a perfect match oligonucleotide and a mismatch oligonucleotide. The perfect match probe has a sequence exactly complimentary to the particular gene and thus measures the expression of the gene. The mismatch probe differs from the perfect match probe by a single base substitution at the center base position, disturbing the binding of the target gene transcript. This helps to determine the background and nonspecific hybridization that contributes to the signal measured for the perfect match oligo. The Microarray Suite software subtracts the hybridization intensities of the mismatch probes from those of the perfect match probes to determine the absolute or specific intensity value for each probe set. Probes are chosen based on current information from Genbank and other nucleotide repositories. The sequences are believed to recognize unique regions of the 3′ end of the gene. A GeneChip Hybridization Oven (“rotisserie” oven) is used to carry out the hybridization of up to 64 arrays at one time. The fluidics station performs washing and staining of the probe arrays. It is completely automated and contains four modules, with each module holding one probe array. Each module is controlled independently through Microarray Suite software using preprogrammed fluidics protocols. The scanner is a confocal laser fluorescence scanner which measures fluorescence intensity emitted by the labeled cRNA bound to the probe arrays. The computer workstation with Microarray Suite software controls the fluidics station and the scanner. Microarray Suite software can control up to eight fluidics stations using preprogrammed hybridization, wash, and stain protocols for the probe array. The software also acquires and converts hybridization intensity data into a presence/absence call for each gene using appropriate algorithms. Finally, the software detects changes in gene expression between experiments by comparison analysis and formats the output into .txt files, which can be used with other software programs for further data analysis.

In some embodiments, the level of at least one mRNA is normalized. In some embodiments, the level of at least two mRNAs are normalized and compared to each other. In some embodiments, such normalization may allow comparison of mRNA levels when the levels are not determined simultaneously and/or in the same assay reaction. One skilled in the art can select a suitable basis for normalization, such as at least one reference mRNA or other factor, depending on the assay.

In some embodiments, at least one reference mRNA comprises a housekeeping gene. In some embodiments, at least one reference mRNA comprises one or more of the normalization genes listed in Table 2. In some embodiments, at least one reference mRNA includes two or more, three or more, four or more, five or more, six or more, seven or more, eight or more, nine or more, ten or more, or all eleven of the normalization genes listed in Table 2. In some embodiments, the first ten genes listed in Table 2 are used. In some embodiments, all eleven genes listed in Table 2 are used.

c. RNA Signature Score Determination

In some embodiments, the RNA signature score is determined for each sample as follows. Raw RNA levels for each gene of the gene signature (Table 1) and a normalization gene set (see, e.g., Table 2; first 10 genes, or all 11 genes) are determined using, e.g., the nCounter® Analysis System (NanoString® Technologies, Seattle, Wash.), and these levels are transformed by log (2) transformation. The arithmetic mean of the log (2) transformed RNA levels of the normalization genes is obtained, and this number is subtracted from the log (2) transformed RNA levels for each of the signature genes, and this value is added to 10. This gives the housekeeper normalized, log 2 transformed value for each signature gene. Next, these values are transformed by using them as exponents (taking 2 to the power of each value) followed by a log 10 transformation to give the unweighted, log 10 transformed housekeeper normalized expression levels. Next, weighting of each signature gene is done by multiplying unweighted, log 10 transformed housekeeper normalized expression levels against the respective factor indicated for each gene in the fourth column of Table 1. A final weighted score is then obtained by adding the weighted numbers for each gene of the signature. In some embodiments, a gene signature is considered to be elevated if it is about 2.97 or above. In some embodiments, a gene signature is considered to be elevated if it is about 3.39 or above. In some embodiments, a gene signature is considered to be elevated if it is 3.40 or is about 3.40 or above. In some embodiments, a gene signature is considered to be elevated if it is about 3.58 or above. In some embodiments, a gene signature is considered to be elevated if it is about 2.97 to 3.58 or above.

It is understood that the method described above is exemplary only and that other approaches can be used to obtain information that is equivalent to that set forth above but is expressed differently. Thus, for example, it is thus understood that using the same genes of the gene signature of Table 1, the same normalization gene set noted above, and weights that are proportionally the same as those set forth in the fourth column of Table 1, but, e.g., a different transformation can be used. The resulting information will be equivalent to that obtained using the methods described above, and thus is included in the present invention even though, e.g., the cut-off numbers will be different.

d. Kits and Compositions

Provided herein are also polynucleotides, kits, medicines, and compositions suitable for use in methods such as those described herein.

In some embodiments, a polynucleotide provided herein is isolated. In some embodiments, a polynucleotide provided herein is detectably labeled, e.g., with a radioisotope, a fluorescent agent, or a chromogenic agent. In another embodiment, a polynucleotide is a primer. In another embodiment, a polynucleotide is an oligonucleotide, e.g., an mRNA-specific oligonucleotide. In another embodiment, an oligonucleotide may be, for example, from 7-60 nucleotides in length, 9-45 nucleotides in length, 15-30 nucleotides in length, or 18-25 nucleotides in length. In another embodiment, an oligonucleotide may be, e.g., PNA, morpholino-phosphoramidates, LNA, or 2′-alkoxyalkoxy. Polynucleotides as provided herein are useful, e.g., for the detection of target sequences, such as sequences contained within the RNA signature or a reference mRNA, such as the reference mRNAs discussed above.

Detection can involve hybridization, amplification, and/or sequencing, as discussed above.

In some embodiments, compositions are provided that include a plurality of polynucleotides, the plurality including at least a first polynucleotide specific for a first mRNA and a second polynucleotide specific for a second mRNA, the first and second mRNAs being selected from the mRNAs in the RNA signature. In some embodiments, the plurality further comprises a third polynucleotide specific for a third mRNA, the third mRNA being selected from the mRNAs in the RNA signature. In some embodiments, the plurality further comprises a fourth polynucleotide specific for a fourth mRNA, the fourth mRNA being selected from the mRNAs in the RNA signature. In some embodiments, the plurality further comprises a fifth polynucleotide specific for a fifth mRNA, the fifth mRNA being selected from the mRNAs in the RNA signature. In some embodiments, the plurality further comprises a sixth polynucleotide specific for a sixth mRNA, the sixth mRNA being selected from the mRNAs in the RNA signature. In some embodiments, the plurality further comprises a seventh polynucleotide specific for a seventh mRNA, the seventh mRNA being selected from the mRNAs in the RNA signature. In some embodiments, the plurality further comprises an eighth polynucleotide specific for an eighth mRNA, the eighth mRNA being selected from the mRNAs in the RNA signature. In some embodiments, the plurality further comprises a ninth polynucleotide specific for a ninth mRNA, the ninth mRNA being selected from the mRNAs in the RNA signature. In some embodiments, the plurality further comprises a tenth polynucleotide specific for a tenth mRNA, the tenth mRNA being selected from the mRNAs in the RNA signature. In some embodiments, the plurality further comprises an eleventh polynucleotide specific for an eleventh mRNA, the eleventh mRNA being selected from the mRNAs in the RNA signature. In some embodiments, the plurality further comprises a twelfth polynucleotide specific for a twelfth mRNA, the twelfth mRNA being selected from the mRNAs in the RNA signature. In some embodiments, the plurality further comprises a thirteenth polynucleotide specific for a thirteenth mRNA, the thirteenth mRNA being selected from the mRNAs in the RNA signature. In some embodiments, the plurality further comprises a fourteenth polynucleotide specific for a fourteenth mRNA, the fourteenth mRNA being selected from the mRNAs in the RNA signature. In some embodiments, the plurality further comprises a fifteenth polynucleotide specific for a fifteenth mRNA, the fifteenth mRNA being selected from the mRNAs in the RNA signature. In some embodiments, the plurality further comprises a sixteenth polynucleotide specific for a sixteenth mRNA, the sixteenth mRNA being selected from the mRNAs in the RNA signature. In some embodiments, the plurality further comprises a seventeenth polynucleotide specific for a seventeenth mRNA, the seventeenth mRNA being selected from the mRNAs in the RNA signature. In some embodiments, the plurality further comprises an eighteenth polynucleotide specific for an eighteenth mRNA, the eighteenth mRNA being selected from the mRNAs in the RNA signature. It is understood that the use of ordinals (“first,” “second,” etc.) to designate polynucleotides or mRNAs indicates that the polynucleotides or mRNAs, as the case may be, are not identical to each other. It is also understood that in embodiments in which the “first,” “second,” etc. polynucleotides are primers (e.g., primers for carrying out an amplification reaction, such as PCR are related methods), the compositions can also optionally include one or more corresponding primers that hybridize to the opposite strand in order to facilitate amplification. It is further understood that in embodiments in which the “first,” “second,” etc. polynucleotides are capture probes, the compositions can also optionally include one or more corresponding detection probes that hybridize to same target in order to facilitate detection and quantitation.

In some embodiments, a composition includes cells or tissue obtained from a subject. In some embodiments, a composition comprises mRNA isolated from a subject. In some embodiments, a composition comprises cDNA synthesized from mRNA isolated from a subject. In some embodiments, a composition includes control cells, tissues, mRNA, or cDNA.

In some embodiments, a composition comprises at least one polynucleotide or a plurality of polynucleotides suitable for use in detecting at least one reference mRNA. In some embodiments, a composition comprises reagents for performing hybridization and/or amplification, such as quantitative RT-PCR, microarray, digital PCR, RNA-Seq, RPA, Northern blot, or in situ hybridization ISH. Such reagents can include one or more of an enzyme with reverse transcriptase activity, a DNA polymerase (which may be thermophilic), an intercalating dye, dNTPs, buffer, a single-strand RNA nuclease, detergent, fixative (e.g., formaldehyde), cosolvent (e.g., formamide), etc.

In some embodiments, a kit is provided including one or more containers comprising at least one polynucleotide specific for an mRNA selected from the mRNAs in the RNA signature or a plurality of polynucleotides, the plurality comprising at least a first polynucleotide specific for a first mRNA and a second polynucleotide specific for a second mRNA, the first and second mRNAs being selected from the mRNAs in the RNA signature. In some embodiments, the plurality further comprises a third polynucleotide specific for a third mRNA, the third mRNA being selected from the mRNAs in the RNA signature. In some embodiments, the plurality further comprises a fourth polynucleotide specific for a fourth mRNA, the fourth mRNA being selected from the mRNAs in the RNA signature. In some embodiments, the plurality further comprises a fifth polynucleotide specific for a fifth mRNA, the fifth mRNA being selected from the mRNAs in the RNA signature. In some embodiments, the plurality further comprises a sixth polynucleotide specific for a sixth mRNA, the sixth mRNA being selected from the mRNAs in the RNA signature. In some embodiments, the plurality further comprises a seventh polynucleotide specific for a seventh mRNA, the seventh mRNA being selected from the mRNAs in the RNA signature. In some embodiments, the plurality further comprises an eighth polynucleotide specific for an eighth mRNA, the eighth mRNA being selected from the mRNAs in the RNA signature. In some embodiments, the plurality further comprises a ninth polynucleotide specific for a ninth mRNA, the ninth mRNA being selected from the mRNAs in the RNA signature. In some embodiments, the plurality further comprises a tenth polynucleotide specific for a tenth mRNA, the tenth mRNA being selected from the mRNAs in the RNA signature. In some embodiments, the plurality further comprises an eleventh polynucleotide specific for an eleventh mRNA, the eleventh mRNA being selected from the mRNAs in the RNA signature. In some embodiments, the plurality further comprises a twelfth polynucleotide specific for a twelfth mRNA, the twelfth mRNA being selected from the mRNAs in the RNA signature. In some embodiments, the plurality further comprises a thirteenth polynucleotide specific for a thirteenth mRNA, the thirteenth mRNA being selected from the mRNAs in the RNA signature. In some embodiments, the plurality further comprises a fourteenth polynucleotide specific for a fourteenth mRNA, the fourteenth mRNA being selected from the mRNAs in the RNA signature. In some embodiments, the plurality further comprises a fifteenth polynucleotide specific for a fifteenth mRNA, the fifteenth mRNA being selected from the mRNAs in the RNA signature. In some embodiments, the plurality further comprises a sixteenth polynucleotide specific for a sixteenth mRNA, the sixteenth mRNA being selected from the mRNAs in the RNA signature. In some embodiments, the plurality further comprises a seventeenth polynucleotide specific for a seventeenth mRNA, the seventeenth mRNA being selected from the mRNAs in the RNA signature. In some embodiments, the plurality further comprises an eighteenth polynucleotide specific for an eighteenth mRNA, the eighteenth mRNA being selected from the mRNAs in the RNA signature. It is understood that the use of ordinals (“first,” “second,” etc.) to designate polynucleotides or mRNAs indicates that the polynucleotides or mRNAs, as the case may be, are not identical to each other. It is also understood that in embodiments in which the “first,” “second,” etc. polynucleotides are primers (e.g., primers for carrying out an amplification reaction, such as PCR are related methods), the kits can also optionally include one or more corresponding primers that hybridize to the opposite strand in order to facilitate amplification. It is further understood that in embodiments in which the “first,” “second,” etc. polynucleotides are capture probes, the kits can also optionally include one or more corresponding detection probes that hybridize to same target in order to facilitate detection and quantitation.

In some embodiments, the kit includes one or more containers comprising at least one polynucleotide or a plurality of polynucleotides suitable for use in detecting at least one reference mRNA. In some embodiments, the kit comprises one or more containers comprising reagents for performing hybridization and/or amplification, such as quantitative RT-PCR, microarray, digital PCR, RNA-Seq, RNase Protection Assay (RPA), Northern blot, and in situ hybridization (ISH). Such reagents can include one or more of an enzyme with reverse transcriptase activity, a DNA polymerase (which may be thermophilic), an intercalating dye, dNTPs, buffer, a single-strand RNA nuclease, detergent, fixative (e.g., formaldehyde), co-solvent (e.g., formamide), etc. The kits of the invention can optionally include control samples to which the RNA signature score of a test sample can be compared in order to determine whether the RNA signature score of the test sample is elevated.

In addition to components for use in detection of RNA signature components, the kits of the invention can also optionally include one or more therapeutic agents for administration to a subject if, e.g., a sample of the subject is found to have an elevated RNA signature score. These therapeutic agents can include one or more ICOS agonists and/or one or more PD-1 antagonists, such as those described herein. These components can optionally be present in the kits in dosage form to facilitate administration. For example, in some embodiments, such a unit dosage is supplied in single-use prefilled syringe for injection. In some embodiments, the composition contained in the unit dosage can include saline, sucrose, or the like; a buffer, such as phosphate, or the like; and/or be formulated within a stable and effective pH range. In some embodiments, the composition can be provided as a lyophilized powder that may be reconstituted upon addition of an appropriate liquid, for example, sterile water. In some embodiments, the composition includes one or more substances that inhibit protein aggregation, including, for example, sucrose and arginine. In some embodiments, a composition includes heparin and/or a proteoglycan. In some embodiments, the amount of the ICOS agonist and/or PD-1 antagonist used in the unit dose can be any of the amounts provided herein for the various methods and/or compositions described.

In some embodiments, kits further include instructions for use in the determination of RNA signature score and, optionally, the treatment of cancer with ICOS agonist and PD-1 antagonist therapy. The kits may further include a description of selection a subject suitable for treatment. Instructions supplied in the kits are typically written instructions on a label or package insert (for example, a paper sheet included in the kit), but machine-readable instructions (for example, instructions carried on a magnetic or optical storage disk) are also acceptable. The kits are in suitable packaging, which may include, for example, vials, bottles, jars, flexible packaging (for example, sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

### III. Methods of Treatment

Subjects having an elevated RNA signature score are administered ICOS agonist and PD-1 antagonist therapy as described herein. This treatment can be used in methods for preventing, improving, or treating cancer in the subjects.

Subjects that can be treated as described herein thus include patients having cancer. The type of cancer can be any type of cancer listed herein or otherwise known in the art. Exemplary types of cancer include, but are not limited to, gastric cancer, breast cancer (e.g., triple negative breast cancer (TNBC)), lung cancer (e.g., non-small cell lung cancer (NSCLC)), melanoma, renal cell carcinoma (RCC), bladder cancer, endometrial cancer, diffuse large B-cell lymphoma (DLBCL), Hodgkin's lymphoma, ovarian cancer, and head and neck squamous cell cancer (HNSCC). Also see the definition of cancer, above, for additional cancer types that can be treated according to the methods of the invention.

Patients that can be treated as described herein include patients who have not previously received a different anti-cancer therapy and patients who have received previous (e.g., 1, 2, 3, 4, 5, or more) doses or cycles of one or more (e.g., 1, 2, 3, 4, 5, or more) of different anti-cancer therapies including, e.g., treatment with one or more ICOS agonists and/or PD-1 antagonist (or any other agent described herein).

The combination treatments of the methods described herein can be concurrent or sequential, as determined to be appropriate by those of skill in the art. Thus, in some embodiments, one or more ICOS agonist (e.g., as described herein) can be administered at the same time as, before, or after one or more PD-1 antagonist (e.g., as described herein). If administered before or after, the administration of the ICOS and PD-1 targeted agents can overlap in time at least in part, and thus be concurrent. Alternatively, the administrations can be such that they do not overlap, and thus be sequential. In other embodiments, one or more PD-1 antagonist (e.g., as described herein) can be administered before or after one or more ICOS agonist (e.g., as described herein), whether concurrently or sequentially.

In addition to treatment with ICOS agonist and PD-1 antagonist therapy, any one or more of the anti-cancer therapies listed herein (see below) and others known in the art can be used in combination with the methods of the invention. In some embodiments, the one or more additional anti-cancer therapies is two or more anti-cancer therapies. In some embodiments, the one or more additional anti-cancer therapies is three or more anti-cancer therapies. Specific, non-limiting examples of additional anti-cancer therapies that can be used in the invention including, e.g., immunotherapies, chemotherapies, and cancer vaccines, among others, are provided below. In some embodiments, the one or more additional anti-cancer therapies is administered prior to the combination therapy of the invention. In some embodiments, the one or more additional anti-cancer therapies is administered at the same time as the combination therapy of the invention. In some embodiments, the one or more additional anti-cancer therapies is administered after the combination therapy of the invention.

In some embodiments, the combination therapy of the invention (and/or the one or more additional anti-cancer therapies) is administered to the patient multiple times at regular intervals. These multiple administrations can also be referred to as administration cycles or therapy cycles. In some embodiments, the combination therapy (and/or the one or more anti-cancer therapies) is administered to the patient for more than two cycles, more than three cycles, more than four cycles, more than five cycles, more than ten cycles, more than fifteen cycles, or more than twenty cycles.

In some embodiments, the regular interval is a dosage every week, a dosage every two weeks, a dosage every three weeks, a dosage every four weeks, a dosage every five weeks, a dosage every six weeks, a dosage every seven weeks, a dosage every eight weeks, a dosage every nine weeks, a dosage every ten weeks, a dosage every eleven weeks, or a dosage every twelve weeks.

### Therapeutic Anti-ICOS Antibodies

Therapeutic anti-ICOS antibodies that can be used in the invention include, but are not limited to, humanized antibodies, chimeric antibodies, human antibodies, and antibodies comprising any of the heavy chain and/or light chain CDRs discussed herein. In some embodiments, the antibody is an isolated antibody. In some embodiments, the antibody is a monoclonal antibody. In some embodiments, the anti-ICOS antibody is an anti-ICOS agonist antibody. See WO 2016/154177 and WO 2017/070423, which are each specifically incorporated herein by reference.

In some embodiments, the therapeutic anti-ICOS agonist antibody includes at least one, two, there, four, five, or all six CDRs selected from (a) HCDR1 comprising the amino acid sequence of SEQ ID NO: 5; (b) HCDR2 comprising the amino acid sequence of SEQ ID NO: 6; (c) HCDR3 comprising the amino acid sequence of SEQ ID NO: 7; (d) LCDR1 comprising the amino acid sequence of SEQ ID NO: 8; (e) LCDR2 comprising the amino acid sequence of SEQ ID NO: 9; and (f) LCDR3 comprising the amino acid sequence of SEQ ID NO: 10. In various embodiments, one or more of the CDRs includes a substitution or deletion that does not destroy specific binding to ICOS. In some embodiments, one or more of the CDRs includes 1, 2, 3, or more substitutions, which may optionally comprise substitutions with conservative amino acids. In some embodiments, one or more of the CDRs includes 1, 2, 3, or more deletions.

In some embodiments, the therapeutic anti-ICOS antibody comprises six CDRs including (a) HCDR1 comprising the amino acid sequence of SEQ ID NO: 5; (b) HCDR2 comprising the amino acid sequence of SEQ ID NO: 6; (c) HCDR3 comprising the amino acid sequence of SEQ ID NO: 7; (d) LCDR1 comprising the amino acid sequence of SEQ ID NO: 8; (e) LCDR2 comprising the amino acid sequence of SEQ ID NO: 9; and (f) LCDR3 comprising the amino acid sequence of SEQ ID NO: 10.

In some embodiments, a therapeutic anti-ICOS antibody comprises a heavy chain variable region and a light chain variable region. In some embodiments, a therapeutic anti-ICOS antibody comprises at least one heavy chain comprising a heavy chain variable region and at least a portion of a heavy chain constant region, and at least one light chain comprising a light chain variable region and at least a portion of a light chain constant region. In some embodiments, a therapeutic anti-ICOS antibody comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some embodiments, the heavy chain is the region of the anti-ICOS antibody that comprises the three heavy chain CDRs. In some embodiments, the light chain is the region of the therapeutic anti-ICOS antibody that comprises the three light chain CDRs.

In some embodiments, the therapeutic anti-ICOS antibody comprises at least one, at least two, or all three VH CDR sequences selected from (a) HCDR1 comprising the amino acid sequence of SEQ ID NO: 5; (b) HCDR2 comprising the amino acid sequence of SEQ ID NO: 6; and (c) HCDR3 comprising the amino acid sequence of SEQ ID NO: 7.

In some embodiments, the therapeutic antibody comprises at least one, at least two, or all three VL CDR sequences selected from (a) LCDR1 comprising the amino acid sequence of SEQ ID NO: 8; (b) LCDR2 comprising the amino acid sequence of SEQ ID NO: 9; and (c) LCDR3 comprising the amino acid sequence of SEQ ID NO: 10.

In some embodiments, the therapeutic anti-ICOS antibody comprises (I) a VH domain comprising at least one, at least two, or all three VH CDR sequences selected from (a) HCDR1 comprising the amino acid sequence of SEQ ID NO: 5; (b) HCDR2 comprising the amino acid sequence of SEQ ID NO: 6; and (c) HCDR3 comprising the amino acid sequence of SEQ ID NO: 7; and (II) a VL domain comprising at least one, at least two, or all three VL CDR sequences selected from (d) LCDR1 comprising the amino acid sequence of SEQ ID NO: 8; (e) LCDR2 comprising the amino acid sequence of SEQ ID NO: 9; and (f) LCDR3 comprising the amino acid sequence of SEQ ID NO: 10.

In some embodiments, a therapeutic anti-ICOS antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 3. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted, and/or deleted in SEQ ID NO: 3. In some embodiments, substitutions, insertions, or deletions occur in regions outside the CDRs (that is, in the FRs). Optionally, the therapeutic anti-ICOS antibody comprises the VH sequence in SEQ ID NO: 3, including post-translational modifications of that sequence.

In some embodiments, the VH comprises: (a) HCDR1 comprising the amino acid sequence of SEQ ID NO: 5; (b) HCDR2 comprising the amino acid sequence of SEQ ID NO: 6; and (c) HCDR3 comprising the amino acid sequence of SEQ ID NO: 7.

In some embodiments, a therapeutic anti-ICOS antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 4. In some embodiments, a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted, and/or deleted in SEQ ID NO: 4. In some embodiments, the substitutions, insertions, or deletions occur in regions outside the CDRs (that is, in the FRs). Optionally, the therapeutic anti-ICOS antibody comprises the VL sequence in SEQ ID NO: 4, including post-translational modifications of that sequence.

In some embodiments, the VL comprises: (a) LCDR1 comprising the amino acid sequence of SEQ ID NO: 8; (b) LCDR2 comprising the amino acid sequence of SEQ ID NO: 9; and (c) LCDR3 comprising the amino acid sequence of SEQ ID NO: 10.

In some embodiments, a therapeutic anti-ICOS antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 3 and a light chain variable domain (VL) having at least 90%, 91%, 92%, 93%, 94%, 95%, 96%, 97%, 98%, 99%, or 100% sequence identity to the amino acid sequence of SEQ ID NO: 4. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, and a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted, and/or deleted in SEQ ID NO: 3. In some embodiments, a total of 1 to 10 amino acids have been substituted, inserted, and/or deleted in SEQ ID NO: 4. In some embodiments, substitutions, insertions, or deletions occur in regions outside the CDRs (that is, in the FRs). Optionally, the therapeutic anti-ICOS antibody comprises the VH sequence in SEQ ID NO: 3 and the VL sequence of SEQ ID NO: 4, including post-translational modifications of one or both sequence.

In some embodiments, the therapeutic anti-ICOS antibody comprises (I) a VH domain comprising: (a) HCDR1 comprising the amino acid sequence of SEQ ID NO: 5; (b) HCDR2 comprising the amino acid sequence of SEQ ID NO: 6; and (c) HCDR3 comprising the amino acid sequence of SEQ ID NO: 7; and (II) a VL domain comprising: (d) LCDR1 comprising the amino acid sequence of SEQ ID NO: 8; (e) LCDR2 comprising the amino acid sequence of SEQ ID NO: 9; and (f) LCDR3 comprising the amino acid sequence of SEQ ID NO: 10.

In some embodiments, a therapeutic anti-ICOS antibody comprises a VH as in any of the embodiments provided herein, and a VL as in any of the embodiments provided herein. In some embodiments, the antibody comprises the VH and VL sequences in SEQ ID NO: 3 and SEQ ID NO: 4, respectively, including post-translational modifications of those sequences.

In some embodiments, a therapeutic anti-ICOS antibody comprises a heavy chain comprising the amino acid sequence of SEQ ID NO: 1, or a variant thereof.

In some embodiments, a therapeutic anti-ICOS antibody comprises a light chain comprising the amino acid sequence of SEQ ID NO: 2, or a variant thereof.

In some embodiments, a therapeutic anti-ICOS antibody comprises a heavy chain comprising the amino acid sequence of SEQ ID NO: 1 and a light chain comprising the amino acid sequence of SEQ ID NO: 2, or variants thereof.

In some embodiments, the therapeutic anti-ICOS antibody comprises the six CDRs as described above and binds to ICOS. In some embodiments, the therapeutic anti-ICOS antibody comprises the six CDRs as described above, binds to ICOS and increases the number of Teff cells and/or activates Teff cells and/or decreases the number of Treg cells and/or increases the ratio of Teff cells to Treg cells in a mammal, such as a human. In some embodiments, the Treg cells are CD4+ FoxP3+ T cells. In some embodiments, the Teff cells are CD8+ T cells. In some embodiments, the Teff cells are CD4+ FoxP3− T cells and/or CD8+ T cells.

Exemplary therapeutic anti-ICOS antibodies include, but are not limited to, JTX-2011 (Jounce Therapeutics; US 2016/0304610; WO 2016/154177; WO 2017/070423) and BMS-986226 (Bristol-Myers Squibb).

In general, therapeutic anti-ICOS antibodies can be administered in an amount in the range of about 10 μg/kg body weight to about 100 mg/kg body weight per dose. In some embodiments, therapeutic anti-ICOS antibodies may be administered in an amount in the range of about 50 μg/kg body weight to about 5 mg/kg body weight per dose. In some embodiments, therapeutic anti-ICOS antibodies may be administered in an amount in the range of about 100 μg/kg body weight to about 10 mg/kg body weight per dose. In some embodiments, therapeutic anti-ICOS antibodies may be administered in an amount in the range of about 100 μg/kg body weight to about 20 mg/kg body weight per dose. In some embodiments, therapeutic anti-ICOS antibodies may be administered in an amount in the range of about 0.5 mg/kg body weight to about 20 mg/kg body weight per dose. In some embodiments, anti-ICOS antibodies may be administered in an amount in the range of about 0.05 mg/kg body weight to about 10 mg/kg body weight per dose. In some embodiments, anti-ICOS antibodies may be administered in an amount in the range of about 5 mg/kg body weight or lower, for example less than 4, less than 3, less than 2, or less than 1 mg/kg of the antibody. In specific examples, therapeutic anti-ICOS antibodies are administered at 0.1 mg/kg, 0.3 mg/kg, or 1.0 mg/kg, once every 3, 6, 9, or 12 weeks.

### PD-1 Therapies

In some embodiments, the one or more anti-cancer therapies is a PD-1 therapy. A PD-1 therapy encompasses any therapy that modulates PD-1 binding to PD-L1 and/or PD-L2. PD-1 therapies may, for example, directly interact with PD-1 and/or PD-L1. In some embodiments, a PD-1 therapy includes a molecule that directly binds to and/or influences the activity of PD-1. In some embodiments, a PD-1 therapy includes a molecule that directly binds to and/or influences the activity of PD-L1. Thus, an antibody that binds to PD-1 or PD-L1 and blocks the interaction of PD-1 to PD-L1 is a PD-1 therapeutic. When a desired subtype of PD-1 therapy is intended, it will be designated by the phrase “PD-1 specific” for a therapy involving a molecule that interacts directly with PD-1, or “PD-L1 specific” for a molecule that interacts directly with PD-L1, as appropriate. Unless designated otherwise, all disclosure contained herein regarding PD-1 therapy applies to PD-1 therapy generally, as well as PD-1 specific and/or PD-L1 specific therapies.

Non-limiting, exemplary PD-1 therapies include nivolumab (OPDIVO®, BMS-936558, MDX-1106, ONO-4538); pidilizumab, lambrolizumab/pembrolizumab (KEYTRUDA, MK-3475); BGB-A317, tislelizumab (BeiGene/Celgene); durvalumab (anti-PD-L1 antibody, MEDI-4736; AstraZeneca/Medlmmune); RG-7446; avelumab (anti-PD-L1 antibody; MSB-0010718C; Pfizer); AMP-224; BMS-936559 (anti-PD-L1 antibody); AMP-514; MDX-1105; A B-011; anti-LAG-3/PD-1; spartalizumab (CoStim/Novartis); anti-PD-1 antibody (Kadmon Pharm.); anti-PD-1 antibody (Immunovo); anti-TEVI-3/PD-I antibody (AnaptysBio); anti-PD-L1 antibody (CoStim/Novartis); RG7446/MPDL3280A (anti-PD-L1 antibody, Genentech/Roche); KD-033 (Kadmon Pharm.); AGEN-2034 (Agenus); STI-A1010; STI-A1110; TSR-042; atezolizumab (TECENTRIQ™); and other antibodies that are directed against programmed death-1 (PD-1) or programmed death ligand 1 (PD-L1).

PD-1 therapies are administered according to regimens that are known in the art, e.g., US FDA-approved regimens. In one example, nivolumab is administered as an intravenous infusion over 60 minutes in the amount of 240 mg every two weeks (unresectable or metastatic melanoma, adjuvant treatment for melanoma, non-small cell lung cancer (NSCLC), advanced renal cell carcinoma, locally advanced renal cell carcinoma, MSI-H or dMMR metastatic colorectal cancer, and hepatocellular carcinoma) or in the amount of 3 mg/kg every three weeks (classical Hodgkin lymphoma; recurrent or metastatic squamous cell carcinoma of the head and neck). In another example, pembrolizumab is administered by intravenous infusion over 30 minutes in the amount of 200 mg, once every three weeks.

In another example, atezolizumab is administered by intravenous infusion over 60 minutes in the amount of 1200 mg every three weeks. In another example, avelumab is administered by intravenous infusion over 60 minutes in the amount of 10 mg/kg every two weeks. In another example, durvalumab is administered by intravenous infusion over 60 minutes in the amount of 10 mg/kg every two weeks.

### IV. Exemplary Anti-Cancer Therapies for Use in Combination with ICOS Agonist and PD-1 Antagonist Treatment

As examples, any anti-cancer therapy listed herein or otherwise known in the art, can be used in combination with ICOS agonist and PD-1 antagonist therapy as described herein or as a pre-treatment or post-treatment. In various examples, the components of a combination are administered according to dosing regimens described herein (e.g., US FDA-approved dosing regimens; see above), or using other regimens determined to be appropriate by those of skill in the art. Exemplary anti-cancer therapies are described below.

a. Immunotherapies

In some embodiments, the one or more anti-cancer therapies is an immunotherapy. The interaction between cancer and the immune system is complex and multifaceted. See de Visser et al., Nat. Rev. Cancer 6:24-37, 2006. While many cancer patients appear to develop an anti-tumor immune response, cancers also develop strategies to evade immune detection and destruction. Recently, immunotherapy has been developed for the treatment and prevention of cancer and other disorders.

Immunotherapy provides the advantage of cell specificity that other treatment modalities lack. As such, methods for enhancing the efficacy of immune based therapies can be clinically beneficial.

i. Anti-CTLA-4 Antagonist Antibodies

In some embodiments, the one or more anti-cancer therapies is an anti-CTLA-4 antagonist antibody. An anti-CTLA-4 antagonist antibody refers to an agent capable of inhibiting the activity of cytotoxic T-lymphocyte-associated protein 4 (CTLA-4), thereby activating the immune system. The CTLA-4 antagonist may bind to CTLA-4 and reverse CTLA-4-mediated immunosuppression. A non-limiting exemplary anti-CTLA-4 antibody is ipilimumab (YERVOY®, BMS), which may be administered according to methods known in the art, e.g., as approved by the US FDA. For example, ipilimumab may be administered in the amount of 3 mg/kg intravenously over 90 minutes every three weeks for a total of 4 doses (unresectable or metastatic melanoma); or at 10 mg/kg intravenously over 90 minutes every three weeks for a total of 4 doses, followed by 10 mg/kg every 12 weeks for up to 3 years or until documented recurrence or unacceptable toxicity (adjuvant melanoma).

ii. OX40 Agonist Antibodies

In some embodiments, the one or more anti-cancer therapies is an agonist anti-OX40 antibody. An OX40 agonist antibody refers to an agent that induces the activity of OX40, thereby activating the immune system and enhancing anti-tumor activity. Non-limiting, exemplary agonist anti-OX40 antibodies are Medi6469, MedImmune, and MOXR0916/RG7888, Roche. These antibodies may be administered according to methods and in regimens determined to be appropriate by those of skill in the art.

iii. TIGIT Antagonists

In some embodiments, the one or more anti-cancer therapies is TIGIT antagonist. A TIGIT antagonist refers to an agent capable of antagonizing or inhibiting the activity of T-cell immunoreceptor with Ig and ITIM domains (TIGIT), thereby reversing TIGIT-mediated immunosuppression. A non-limiting exemplary TIGIT antagonist is BMS-986207 (Bristol-Myers Squibb/Ono Pharmaceuticals). These agents may be administered according to methods and in regimens determined to be appropriate by those of skill in the art.

iv. IDO Inhibitors

In some embodiments, the one or more anti-cancer therapies is an IDO inhibitor. An IDO inhibitor refers to an agent capable of inhibiting the activity of indoleamine 2,3-dioxygenase (IDO) and thereby reversing IDO-mediated immunosuppression. The IDO inhibitor may inhibit IDO1 and/or ID02 (INDOL1). An IDO inhibitor may be a reversible or irreversible IDO inhibitor. A reversible IDO inhibitor is a compound that reversibly inhibits IDO enzyme activity either at the catalytic site or at a non-catalytic site while an irreversible IDO inhibitor is a compound that irreversibly inhibits IDO enzyme activity by forming a covalent bond with the enzyme. Non-limiting exemplary IDO inhibitors are described, e.g., in US 2016/0060237; and US 2015/0352206. Non-limiting exemplary IDO inhibitors include Indoximod (New Link Genetics), INCB024360 (Incyte Corp), 1-methyl-D-tryptophan (New Link Genetics), and GDC-0919/navoximod (Genentech/New Link Genetics). These agents may be administered according to methods and in regimens determined to be appropriate by those of skill in the art.

v. RORγ Agonists

In some embodiments, the one or more anti-cancer therapies is a RORγ agonist. RORγ agonists refer to an agent capable of inducing the activity of retinoic acid-related orphan receptor gamma (RORγ), thereby decreasing immunosuppressive mechanisms. Non-limiting exemplary RORγ agonists include, but are not limited to, LYC-55716 (Lycera/Celgene) and INV-71 (Innovimmune). These agents may be administered according to methods and in regimens determined to be appropriate by those of skill in the art.

b. Chemotherapies

In some embodiments, the one or more anti-cancer therapies is a chemotherapeutic agent. Exemplary chemotherapeutic agents that can be used include, but are not limited to, capecitabine, cyclophosphamide, dacarbazine, temozolomide, cyclophosphamide, docetaxel, doxorubicin, daunorubicin, cisplatin, carboplatin, epirubicin, eribulin, 5-FU, gemcitabine, irinotecan, ixabepilone, methotrexate, mitoxantrone, oxaliplatin, paclitaxel, nab-paclitaxel, ABRAXA E® (protein-bound paclitaxel), pemetrexed, vinorelbine, vincristine, erlotinib, afatinib, gefitinib, crizotinib, dabrafenib, trametinib, vemurafenib, and cobimetanib. These agents may be administered according to methods and in regimens determined to be appropriate by those of skill in the art.

c. Cancer Vaccines

In some embodiments, the one or more anti-cancer therapies is a cancer vaccine. Cancer vaccines have been investigated as a potential approach for antigen transfer and activation of dendritic cells. In particular, vaccination in combination with immunologic checkpoints or agonists for co-stimulatory pathways have shown evidence of overcoming tolerance and generating increased anti-tumor response. A range of cancer vaccines have been tested that employ different approaches to promoting an immune response against the tumor (see, e.g., Emens L A, Expert Opin Emerg Drugs 13(2): 295-308 (2008)). Approaches have been designed to enhance the response of B cells, T cells, or professional antigen-presenting cells against tumors. Exemplary types of cancer vaccines include, but are not limited to, peptide-based vaccines that employ targeting distinct tumor antigens, which may be delivered as peptides/proteins or as genetically-engineered DNA vectors, viruses, bacteria, or the like; and cell biology approaches, for example, for cancer vaccine development against less well-defined targets, including, but not limited to, vaccines developed from patient-derived dendritic cells, autologous tumor cells or tumor cell lysates, allogeneic tumor cells, and the like.

Exemplary cancer vaccines include, but are not limited to, dendritic cell vaccines, oncolytic viruses, tumor cell vaccines, etc. In some embodiments, such vaccines augment the anti-tumor response. Examples of cancer vaccines also include, but are not limited to, MAGE3 vaccine (e.g., for melanoma and bladder cancer), MUC1 vaccine (e.g., for breast cancer), EGFRv3 (such as Rindopepimut, e.g., for brain cancer, including glioblastoma multiforme), or ALVAC-CEA (e.g., for CEA+ cancers).

Non-limiting exemplary cancer vaccines also include Sipuleucel-T, which is derived from autologous peripheral-blood mononuclear cells (PBMCs) that include antigen-presenting cells (see, e.g., Kantoff P W et al., N Engl J Med. 363:411-22 (2010)). In Sipuleucel-T generation, the patient's PBMCs are activated ex vivo with PA2024, a recombinant fusion protein of prostatic acid phosphatase (a prostate antigen) and granulocyte-macrophage colony-stimulating factor (an immune-cell activator). Another approach to a candidate cancer vaccine is to generate an immune response against specific peptides mutated in tumor tissue, such as melanoma (see, e.g., Carreno et al., Science 348:6236, 2015). Such mutated peptides may, in some embodiments, be referred to as neoantigens. As a non-limiting example of the use of neoantigens in tumor vaccines, neoantigens in the tumor predicted to bind the major histocompatibility complex protein HLA-A*02:01 are identified for individual patients with a cancer, such as melanoma. Dendritic cells from the patient are matured ex vivo, then incubated with neoantigens. The activated dendritic cells are then administered to the patient. In some embodiments, following administration of the cancer vaccine, robust T-cell immunity against the neoantigen is detectable.

In some such embodiments, the cancer vaccine is developed using a neoantigen. In some embodiments, the cancer vaccine is a DNA vaccine. In some embodiments, the cancer vaccine is an engineered virus comprising a cancer antigen, such as PROSTVAC (rilimogene galvacirepvec/rilimogene glafolivec). In some embodiments, the cancer vaccine comprises engineered tumor cells, such as GVAX, which is a granulocyte-macrophage colony-stimulating factor (GM-CSF) gene-transfected tumor cell vaccine (see, e.g., Nemunaitis, Expert Rev. Vaccines 4:259-274, 2005).

The vaccines may be administered according to methods and in regimens determined to be appropriate by those of skill in the art.

d. Additional Exemplary Anti-Cancer Therapies

Further non-limiting, exemplary anti-cancer therapies include Luspatercept (Acceleron Pharma/Celgene); Motolimod (Array BioPharma/Celgene/VentiRx Pharmaceuticals/Ligand); GI-6301 (Globelmmune/Celgene/NantWorks); GI-6200 (Globelmmune/Celgene/NantWorks); BLZ-945 (Celgene/Novartis); ARRY-382 (Array BioPharma/Celgene), or any of the anti-cancer therapies provided in Table 3. These agents may be administered according to methods and in regimens determined to be appropriate by those of skill in the art. In some embodiments, the one or more anti-cancer therapies includes surgery and/or radiation therapy. Accordingly, the anti-cancer therapies can optionally be utilized in the adjuvant or neoadjuvant setting.

### V. Pharmaceutical Compositions and Dosing

Compositions including an ICOS agonist, a PD-1 antagonist, or a combination thereof (or one or more additional anti-cancer therapies as described herein) are provided in formulations with a wide variety of pharmaceutically acceptable carriers, as determined to be appropriate by those of skill in the art (see, for example, Gennaro, Remington: The Science and Practice of Pharmacy with Facts and Comparisons: Drugfacts Plus, 20^{th} ed. (2003); Ansel et al., Pharmaceutical Dosage Forms and Drug Delivery Systems, 7^{th} ed., Lippincott, Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

Anti-cancer therapies are administered in the practice of the methods of the present invention as is known in the art (e.g., according to FDA-approved regimens) or as indicated elsewhere herein (see, e.g., above). In some embodiments, anti-cancer therapies of the invention are administered in amounts effective for treatment of cancer. The therapeutically effective amount is typically dependent on the weight of the subject being treated, his or her physical or health condition, the extensiveness of the condition to be treated, the age of the subject being treated, pharmaceutical formulation methods, and/or administration methods (e.g., administration time and administration route).

In some embodiments, anti-cancer therapies can be administered in vivo by various routes, including, but not limited to, intravenous, intra-arterial, parenteral, intratumoral, intraperitoneal, or subcutaneous. The appropriate formulation and route of administration can be selected by those of skill in the art according to the intended application.

### VI. Examples

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

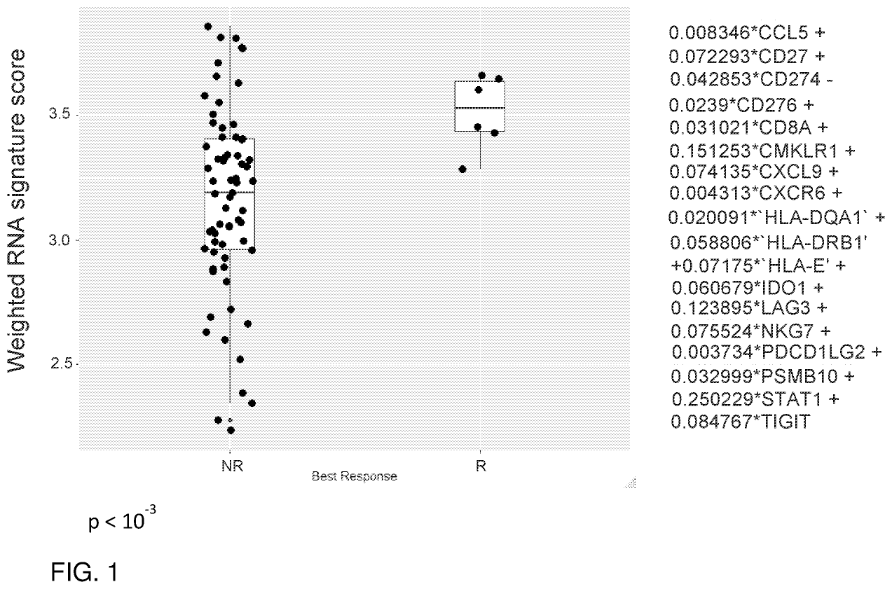

RNA samples were analyzed for levels of the RNAs listed in Table 1 and an RNA signature score was obtained. FIG. 1 shows that the weighted RNA signature score predicts tumor response.

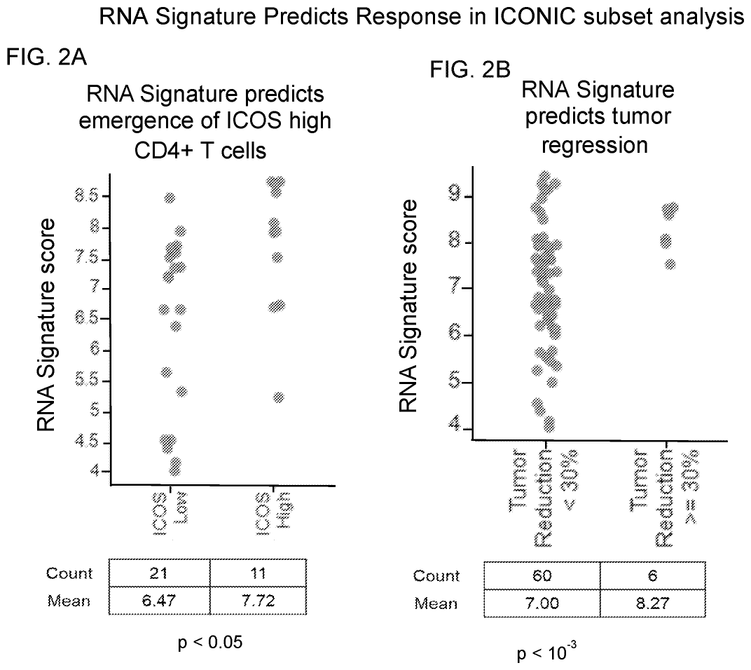

RNA was extracted from fresh pre-treatment tumor samples, gene expression was evaluated by NanoString, and RNA signature was calculated, as described above. ICOS hi emergence was evaluated on subject-matched peripheral blood samples, as described (Hanson et al, Emergence of an ICOS hi CD4 T cell subset correlates with tumor reductions in subjects treated with the ICOS agonist antibody JTX-2011, SITC 2018 poster) and subjects were classified as “ICOS hi” (those that display sustained emergence of an ICOS hi CD4 T cell population) or “ICOS low” (subjects that do not display sustained emergence of an ICOS hi CD4 T cell population). The distribution of RNA signature scores was compared between the two groups using a Welch 2 sample t-test, using the function “t.test” in R (FIG. 2A). The mean values of 6.47 and 7.72 indicated in the figure correspond to RNA signature scores as described elsewhere herein of 2.97 and 3.35, respectively. The distribution in RNA signature scores was compared between subjects classified as responders/non-responders to treatment based on percent tumor reduction. Subjects displaying at least 30% tumor reduction were classified as responders; subjects displaying less than 30% tumor reduction are classified as non-responders. Statistics were calculated using a Welch 2 sample t-test, using the function “t.test” in R (FIG. 2B). The mean values of 7.00 and 8.27 indicated in the figure correspond to RNA signature scores as described elsewhere herein of 3.13 and 3.51, respectively. The results show that RNA signature predicts response in ICONIC subset analysis.

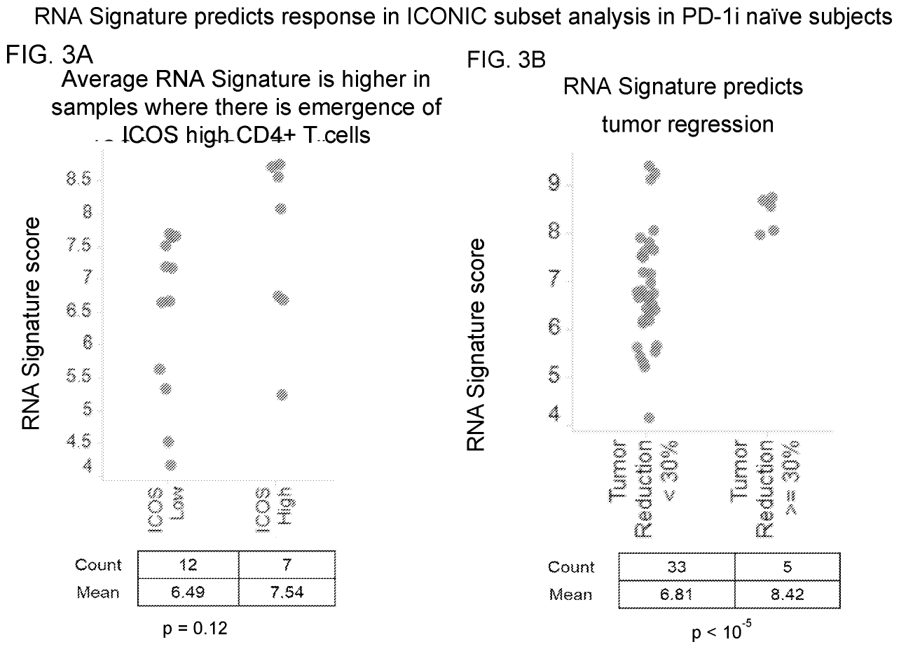

The same experiments described above were carried out with subjects who had not seen prior anti-PD-1 or anti-PD-L1 therapy (FIGS. 3A and 3B). The mean values of 6.49 and 7.54 shown in FIG. 3A correspond to RNA signature scores as described elsewhere herein of 2.98 and 3.29, respectively. The mean values of 6.81 and 8.42 shown in FIG. 3B correspond to RNA signature scores as described elsewhere herein of 3.07 and 3.56, respectively. The results show that RNA signature predicts response on ICONIC subset analysis in these subjects as well.

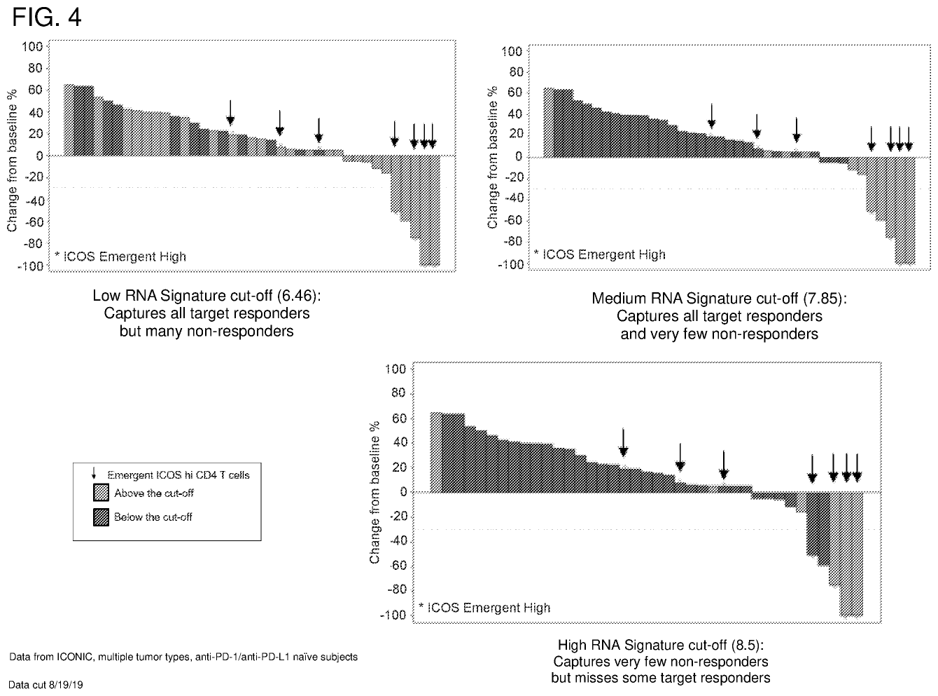

Tumor reductions for all prior anti-PD-1/anti-PD-L1 naïve subjects that had fresh pre-treatment tumor biopsies were evaluated for the RNA signature score (FIG. 4). Bars are shaded by whether the subject's tumor sample is biomarker positive or negative, based on varying thresholds of the RNA signature score. The biomarker threshold is lowest in the left panel (6.46); medium in the middle panel (7.85); and high in the right panel (8.5). The low, medium, and high RNA signature score cut-offs of 6.46, 7.85, and 8.5 indicted in FIG. 4 correspond to RNA signature scores of as described elsewhere herein of 2.97, 3.39, and 3.58, respectively.

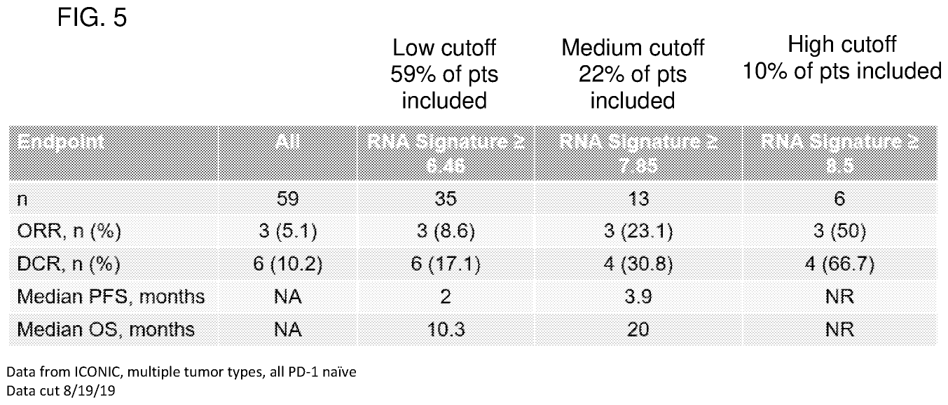

Clinical endpoints were calculated for all prior anti-PD-1/anti-PD-L1 naïve subjects that had fresh pre-treatment tumor biopsies evaluated for the RNA signature score (FIG. 5). The low, medium, and high RNA signature score cut-offs of 6.46, 7.85, and 8.5 indicated in FIG. 5 correspond to RNA signature scores of 2.97, 3.39, and 3.58, respectively, as described elsewhere herein.

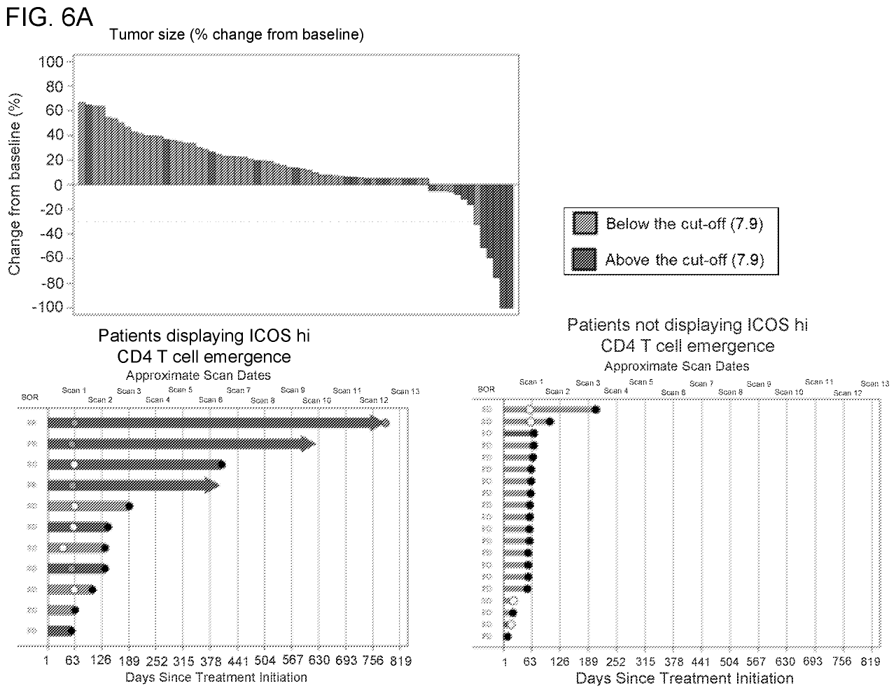

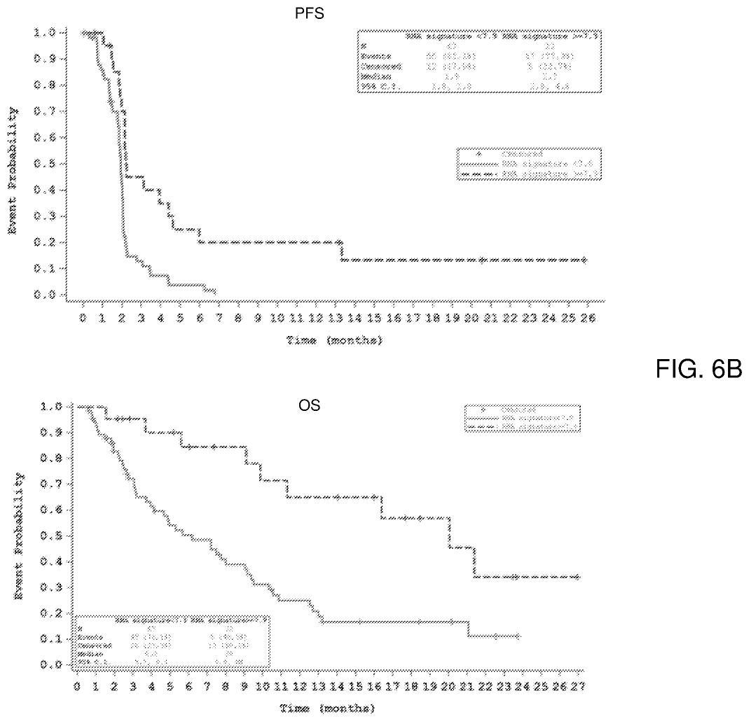

Tumor reductions and swimmers plots for all subjects who had fresh pre-treatment tumor biopsies evaluated for the RNA signature score are shown in FIG. 6A. Bars are shaded by whether the subject's sample is biomarker positive or negative, based on an RNA signature threshold=7.9. Swimmers plots are shown only for subjects who had RNA signature score evaluated on pre-treatment tumor samples and ICOS emergence evaluated on peripheral CD4 T cells over the course of treatment. Kaplan Meier plots of clinical endpoints Progression Free Survival (PFS) and Overall Survival (OS), evaluated for subjects who had fresh pre-treatment tumor samples evaluated for the RNA signature score, are shown in FIG. 6B. Subjects are classified as biomarker positive or negative, based on whether their tumor RNA signature score is greater than or equal to the threshold 7.9. The cut-off of 7.9 described with respect to FIGS. 6A and 6B corresponds to an RNA signature score of 3.40 as described elsewhere herein.

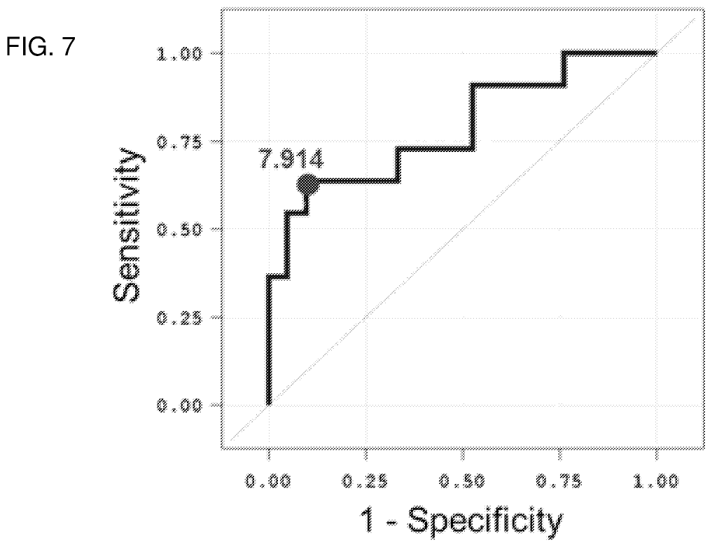

Receiver Operating Characteristic (ROC) curve showing the relationship between the sensitivity and specificity of the RNA signature threshold is shown in FIG. 7. ICOS hi emergence (high, low) is the binary result variable and RNA signature is the predicative variable with various cuts. The optimal cutoff as determined by the Youden index is calculated to be 7.914, which maximizes the difference between the true positive and the false positive rate. These calculations were performed in SAS using the function “PROC LOGISTIC.” The cut-off of 7.914 corresponds to an RNA signature score of 3.40 as described elsewhere herein. The positive predictive value using the optimal cutoff is 78% and the negative predictive value using this cutoff is 83%, using ICOS hi emergence as the response output. The Area Under the Curve (AUC) for the ROC curve is 0.79. ROC curve AUC values can range from 0 to 1; a biomarker is considered to be a random predictor if the AUC of the ROC curve is 0.5. Thus, the cutoff selected has been optimized for both positive and negative predictive power with respect to identifying patients likely to exhibit NCOS hi 004 positive T-cell emergence in response to the combination ICOS agonist and PD1 antagonist therapy described herein.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

TABLE 1
RNA Score Signature Set

| | | Exemplary Target | Scoring |
|---|---|---|---|
| Gene Symbol | Accession No. | Region | Weight |
| CCL5 | NM_002985.2 | 280-380 | 0.008346 |
| CD27 | NM_001242.4 | 330-340 | 0.072293 |
| CD274 | NM_014143.3 | 1245-1345 | 0.042853 |
| CD276 | NM_001024736.1 | 2120-2220 | −0.0239 |
| CD8A | NM_001768.5 | 1320-1420 | 0.031021 |
| CMKLRI | NM_004072.1 | 770-870 | 0.151253 |
| CXCL9 | NM_002416.1 | 1975-2075 | 0.074135 |
| CXCR6 | NM_006564.1 | 95-195 | 0.004313 |
| HLA-DQA1 | NM_002122.3 | 261-361 | 0.020091 |
| HLA-DRBI | NM_002124.1 | 985-1085 | 0.058806 |
| HLA-E | NM_005516.4 | 1204-1304 | 0.07175 |
| IDOl | NM_002164.3 | 50-150 | 0.060679 |
| LAG3 | NM_002286.5 | 1735-1835 | 0.123895 |
| NKG7 | NM_005601.3 | 632-732 | 0.075524 |
| PDCD1LG2 | NM_025239.3 | 235-335 | 0.003734 |
| PSMB10 | NM_002801.2 | 221-321 | 0.032999 |
| STAT1 | NM_007315.2 | 205-305 | 0.250229 |
| TIG IT | NM_173799.2 | 1968-2068 | 0.084767 |

TABLE 2
Normalization Gene Set

| | | Exemplary Target |
|---|---|---|
| Gene Symbol | Accession No. | Region |
| ABCF1 | NM_001090.2 | 850-950 |
| C14ORF102 | NM_017970.3 | 3236-3336 |
| G6PD | NM_000402.2 | 1155-1255 |
| OAZI | NM_004152.2 | 313-413 |
| POLR2A | NM_000937.2 | 3775-3875 |
| SDHA | NM_004168.1 | 230-330 |
| STK11IP | NM_052902.2 | 565-665 |
| TBC1D10B | NM_015527.3 | 2915-3015 |
| TBP | NM_001172085.1 | 587-687 |
| UBB | NM_018955.2 | 795-895 |
| ZBTB34 | NM_001099270.1 | 406-506 |

TABLE 3
Cancer Therapies

| Anti-Cancer | | Anti-Cancer | |
|---|---|---|---|
| Therapeutic | Target Name | Therapeutic | Target Name |
| BMS-986179 | 5'-nucleotidase, ecto | imalumab | macrophage migration |
| | (CD73) | | inhibitory factor |
| | | | (glycosylation-inhibiting |
| | | | factor) |
| pTVG-HP | acid phosphatase, prostate | OSE-2101 | major histocompatibility |
| | | | complex, class I, A |
| sipuleucel-T | acid phosphatase, prostate | andecaliximab | matrix metallopeptidase 9 |
| | | | (gelatinase B, 92kDa |
| | | | gelatinase, 92kDa type IV |
| | | | collagenase) |
| CX-2009 | activated leukocyte cell | anti-MAGE-A3 | melanoma antigen family A, 3 |
| | adhesion molecule | TCR, Kite Pharma | |
| luspatercept | activin A receptor type Il- | KITE-718 | melanoma antigen family A, 3 |
| | like 1 | | |
| CPI-444 | adenosine A2a receptor | biropepimut-S | melanoma antigen family A, 3 |
| NGR-TNF | alanyl (membrane) | rituximab | membrane-spanning 4- |
| | aminopeptidase | biosimilar, Pfizer | domains, subfamily A, |
| | | | member 1 |
| CB-1158 | arginase 1 | rituximab | membrane-spanning 4- |
| | arginase 2 | biosimilar, Dr. | domains, subfamily A, |
| | | Reddy's | member 1 |
| BA3011 | AXL receptor tyrosine | rituximab | membrane-spanning 4- |
| | kinase | biosimilar, Sandoz | domains, subfamily A, |
| | | | member 1 |
| AXL-107-MMAE | AXL receptor tyrosine | rituximab | membrane-spanning 4- |
| | kinase | biosimilar, Celltrion | domains, subfamily A, |
| | | | member 1 |
| CCT301-38 | AXL receptor tyrosine | rituximab | membrane-spanning 4- |
| | kinase | biosimilar, Archigen | domains, subfamily A, |
| | RAR-related orphan | Biotech | member 1 |
| | receptor A | | |
| SurVaxM | baculoviral lAP repeat | rituximab | membrane-spanning 4- |
| | containing 5 | biosimilar, Innovent | domains, subfamily A, |
| | | Biologies | member 1 |
| NY-ESO-1 TCR, | cancer/testis antigen 1 | MB-106 | membrane-spanning 4- |
| Adaptimmune | | | domains, subfamily A, |
| | | | member 1 |
| CDX-1401 | cancer/testis antigen 1 | ibritumomab | membrane-spanning 4- |
| | lymphocyte antigen 75 | tiuxetan | domains, subfamily A, |
| | | | member 1 |
| ETBX-011 | carcinoembryonic antigen- | rituximab | membrane-spanning 4- |
| | related cell adhesion | | domains, subfamily A, |
| | molecule 5 | | member 1 |
| GI-6207 | carcinoembryonic antigen- | ublituximab | membrane-spanning 4- |
| | related cell adhesion | | domains, subfamily A, |
| | molecule 5 | | member 1 |
| falimarev + | carcinoembryonic antigen- | rituximab | membrane-spanning 4- |
| inalimarev | related cell adhesion | biosimilar, | domains, subfamily A, |
| | molecule 5 | Allergan/Amgen | member 1 |
| | mucin 1, cell surface | | |
| | associated | | |
| labetuzumab | carcinoembryonic antigen- | ofatumumab | membrane-spanning 4- |
| govitecan | related cell adhesion | | domains, subfamily A, |
| | molecule 5 | | member 1 |
| | topoisomerase (DNA) I | | |
| coltuximab | CD19 molecule | ocaratuzumab | membrane-spanning 4- |
| ravtansine | | | domains, subfamily A, |
| | | | member 1 |
| denintuzumab | CD19 molecule | veltuzumab | membrane-spanning 4- |
| mafodotin | | | domains, subfamily A, |
| | | | member 1 |
| axicabtagene | CD19 molecule | obinutuzumab | membrane-spanning 4- |
| ciloleucel | | | domains, subfamily A, |
| | | | member 1 |
| CIK-CAR.CD19 | CD19 molecule | rituximab and | membrane-spanning 4- |
| | | hyaluronidase | domains, subfamily A, |
| | | human | member 1 |
| JCAR014 | CD19 molecule | anetumab | mesothelin |
| | | ravtansine | |
| lisocabtagene | CD19 molecule | amatuximab | mesothelin |
| maraleucel | | | |
| tisagenlecleucel | CD19 molecule | emibetuzumab | met proto-oncogene |
| MOR-208 | CD19 molecule | binimetinib | mitogen-activated protein |
| | | | kinase kinase 1 |
| | | | mitogen-activated protein |
| | | | kinase kinase 2 |
| inebilizumab | CD19 molecule | SAR566658 | mucin 1, cell surface |
| | | | associated |
| AUTO3, Autolus | CD19 molecule | Cvac, Prima | mucin 1, cell surface |
| | CD22 molecule | Biomed | associated |
| DT2219ARL | CD19 molecule | TG4010 | mucin 1, cell surface |
| | CD22 molecule | | associated |
| | | | interleukin 2 receptor, alpha |
| blinatumomab | CD19 molecule | oregovomab | mucin 16, cell surface |
| | CD3e molecule, epsilon | | associated |
| | (CD3-TCR complex) | | |
| samalizumab | CD200 molecule | methionine | opioid growth factor receptor |
| | | enkephalin based | |
| | | immunotherapy | |
| inotuzumab | CD22 molecule | olaratumab | platelet-derived growth factor |
| ozogamicin | | | receptor, alpha polypeptide |
| 90Y- | CD22 molecule | enfortumab vedotin | poliovirus receptor-related 4 |
| epratuzumab | | | |
| tetraxetan | | | |
| epratuzumab | CD22 molecule | ProstAtak, | polymerase (DNA directed), |
| | | Advantagene | alpha 1, catalytic subunit |
| ontuxizumab | CD248 molecule, | PancAtak, | polymerase (DNA directed), |
| | endosialin | Advantagene | alpha 1, catalytic subunit |
| varlilumab | CD27 molecule | aglatimagene | polymerase (DNA directed), |
| | | besadenovec | alpha 1, catalytic subunit |
| durvalumab | CD274 molecule | IMC-gp100 | premelanosome protein |
| avelumab | CD274 molecule | cemiplimab | programmed cell death 1 |
| atezolizumab | CD274 molecule | AGEN2034 | programmed cell death 1 |
| CX-072 | CD274 molecule | nivolumab | programmed cell death 1 |
| enoblituzumab | CD276 molecule | pembrolizumab | programmed cell death 1 |
| omburtamab | CD276 molecule | spartalizumab | programmed cell death 1 |
| AlloStim, | CD28 molecule | BGB-A317 | programmed cell death 1 |
| Immunovative | | | |
| Therapies | | | |
| gemtuzumab | CD33 molecule | genolimzumab | programmed cell death 1 |
| ozogamicin | | | |
| lintuzumab- | CD33 molecule | JNJ-63723283 | programmed cell death 1 |
| Ac225 | | | |
| Bl 836858 | CD33 molecule | MEDI0680 | programmed cell death 1 |
| naratuximab | CD37 molecule | thymalfasin | prothymosin, alpha |
| emtansine | | | |
| lutetium (177Lu) | CD37 molecule | LYC-55716 | RAR-related orphan receptor |
| lilotomab | | | c |
| satetraxetan | | | |
| otlertuzumab | CD37 molecule | cirmtuzumab | receptor tyrosine kinase-like |
| | | | orphan receptor 1 |
| daratumumab | CD38 molecule | VX15/2503 | sema domain, |
| | | | immunoglobulin domain (Ig), |
| | | | transmembrane domain (TM) |
| | | | and short cytoplasmic |
| | | | domain, (semaphorin) 4D |
| isatuximab | CD38 molecule | elotuzumab | SLAM family member 7 |
| TAK-573 | CD38 molecule | indatuximab | syndecan 1 |
| | | ravtansine | |
| A-dmDT390- | CD3e molecule, epsilon | BMS-986207 | T-cell immunoreceptor with Ig |
| bisFv (UCHT1) | (CD3-TCR complex) | | and ITIM domains |
| APX005M | CD40 molecule, TNF | tertomotide | telomerase reverse |
| | receptor superfamily | | transcriptase |
| | member 5 | | |
| Hu5F9-G4 | CD47 molecule | Toca 511 + Toca | thymidylate synthetase |
| | | FC | |
| TI-061 | CD47 molecule | APS001F | thymidylate synthetase |
| milatuzumab | CD74 molecule, major | JCARH125 | TNF receptor superfamily |
| | histocompatibility complex, | | member 17 |
| | class II invariant chain | | |
| polatuzumab | CD79b molecule, | bb2121 | TNF receptor superfamily |
| vedotin | immunoglobulin-associated | | member 17 |
| | beta | | |
| mogamulizumab | chemokine (C-C motif) | AUTO2, Autolus | TNF receptor superfamily |
| | receptor 4 | | member 17 |
| | | | TNF receptor superfamily |
| | | | member 13B |
| BL-8040 | chemokine (C-X-C motif) | OPN-305 | toll-like receptor 2 |
| | receptor 4 | | |
| X4P-001 | chemokine (C-X-C motif) | rintatolimod | toll-like receptor 3 |
| | receptor 4 | | |
| ulocuplumab | chemokine (C-X-C motif) | poly-ICLC | toll-like receptor 3 |
| | receptor 4 | | |
| elaud iximab | claudin 18 | ID-G100 | toll-like receptor 4 |
| ALT-836 | coagulation factor III | ID-CMB305 | toll-like receptor 4 |
| | (thromboplastin, tissue | | cancer/testis antigen 1 |
| | factor) | | |
| MCS110 | colony stimulating factor 1 | imiquimod | toll-like receptor 7 |
| | (macrophage) | (intravesical), | |
| | | Telormedix | |
| ARRY-382 | colony stimulating factor 1 | NKTR-262 | toll-like receptor 7 |
| | (macrophage) | | toll-like receptor 8 |
| | colony stimulating factor 1 | | |
| | receptor | | |
| BLZ-945 | colony stimulating factor 1 | motolimod | toll-like receptor 8 |
| | receptor | | |
| AMG 820 | colony stimulating factor 1 | tilsotolimod | toll-like receptor 9 |
| | receptor | | |
| cabiralizumab | colony stimulating factor 1 | sacituzumab | topoisomerase (DNA) I |
| | receptor | govitecan | tumor-associated calcium |
| | | | signal transducer 2 |
| gemogenovatucel-T | colony stimulating factor 2 | HPV-16 E6 TCR, | transforming protein E6, |
| | (granulocyte-macrophage) | Bluebird Bio/Kite | human papilloma virus-16 |
| | | Pharma | |
| GVAX | colony stimulating factor 2 | VGX-3100 | transforming protein E6, |
| | (granulocyte-macrophage) | | human papilloma virus-16 |
| | | | transforming protein E7, |
| | | | human papilloma virus-16 |
| | | | E6 protein, human papilloma |
| | | | virus-18 |
| | | | E7 protein, human papilloma |
| | | | virus-18 |
| talimogene | colony stimulating factor 2 | MEDI0457 | transforming protein E6, |
| laherparepvec | (granulocyte-macrophage) | | human papilloma virus-16 |
| | | | transforming protein E7, |
| | | | human papilloma virus-16 |
| | | | E7 protein, human papilloma |
| | | | virus-18 |
| | | | E6 protein, human papilloma |
| | | | virus-18 |
| pexastimogene | colony stimulating factor 2 | TVGV-1 | transforming protein E7, |
| devacirepvec | (granulocyte-macrophage) | | human papilloma virus-16 |
| sargramostim | colony stimulating factor 2 | KITE-439 | transforming protein E7, |
| | receptor, alpha, low-affinity | | human papilloma virus-16 |
| | (granulocyte-macrophage) | | |
| SV-BR-1-GM | colony stimulating factor 2 | ADXS-DUAL | transforming protein E7, |
| cancer vaccine | receptor, alpha, low-affinity | | human papilloma virus-16 |
| | (granulocyte-macrophage) | | |
| pamrevlumab | connective tissue growth | axalimogene | transforming protein E7, |
| | factor | filolisbac | human papilloma virus-16 |
| ipilimumab | cytotoxic T-lymphocyte- | MVA-5T4 | trophoblast glycoprotein |
| | associated protein 4 | | |
| tremelimumab | cytotoxic T-lymphocyte- | oportuzumab | tumor-associated calcium |
| | associated protein 4 | monatox | signal transducer 2 |
| BMS-986249 | cytotoxic T-lymphocyte- | denosumab | tumour necrosis factor |
| | associated protein 4 | | (ligand) superfamily, member |
| | | | 11 |
| rovalpituzumab | delta-like 3 (Drosophila) | BION-1301 | tumour necrosis factor |
| tesirine | | | (ligand) superfamily, member |
| | | | 13 |
| ABT-165 | delta-like 4 (Drosophila) | belimumab | tumour necrosis factor |
| | vascular endothelial growth | | (ligand) superfamily, member |
| | factor A | | 13b |
| BHQ880 | dickkopf WNT signaling | INCAGN1876 | tumour necrosis factor |
| | pathway inhibitor 1 | | receptor superfamily, member |
| | | | 18 |
| DKN-01 | dickkopf WNT signaling | BMS-986156 | tumour necrosis factor |
| | pathway inhibitor 1 | | receptor superfamily, member |
| | | | 18 |
| Ad-REIC vaccine, | dickkopf WNT signaling | INCAGN1949 | tumour necrosis factor |
| Momotaro-Gene | pathway inhibitor 3 | | receptor superfamily, member |
| | | | 4 |
| AGS-16C3F | ectonucleotide | PF-04518600 | tumour necrosis factor |
| | pyrophosphatase/phosphod | | receptor superfamily, member |
| | iesterase 3 | | 4 |
| carotuximab | endoglin | BMS-986178 | tumour necrosis factor |
| | | | receptor superfamily, member |
| | | | 4 |
| ifabotuzumab | EPH receptor A3 | brentuximab | tumour necrosis factor |
| | | vedotin | receptor superfamily, member |
| | | | 8 |
| CimaVax EGF | epidermal growth factor | urelumab | tumour necrosis factor |
| | (beta-urogastrone) | | receptor superfamily, member |
| | | | 9 |
| depatuxizumab | epidermal growth factor | utomilumab | tumour necrosis factor |
| mafodotin | receptor | | receptor superfamily, member |
| | | | 9 |
| RM-1929 | epidermal growth factor | VBI-1901 | UL83, cytomegalovirus |
| | receptor | | UL55, cytomegalovirus |
| AVID100 | epidermal growth factor | bevacizumab | vascular endothelial growth |
| | receptor | biosimilar, | factor A |
| | | Boehringer | |
| | | Ingelheim | |
| trastuzumab | epidermal growth factor | bevacizumab-awwb | vascular endothelial growth |
| biosimilar, | receptor | | factor A |
| Henlius | | | |
| cetuximab | epidermal growth factor | bevacizumab | vascular endothelial growth |
| | receptor | biosimilar, Pfizer | factor A |
| panitumumab | epidermal growth factor | bevacizumab | vascular endothelial growth |
| | receptor | biosimilar, | factor A |
| | | Oncobiologics | |
| necitumumab | epidermal growth factor | bevacizumab | vascular endothelial growth |
| | receptor | biosimilar, Henlius | factor A |
| | | Biopharmaceuticals | |
| nimotuzumab | epidermal growth factor | bevacizumab | vascular endothelial growth |
| | receptor | biosimilar, Fujifilm | factor A |
| | | Kyowa Kirin | |
| | | Biologies | |
| futuximab | epidermal growth factor | aflibercept | vascular endothelial growth |
| | receptor | | factor A |
| tomuzotuximab | epidermal growth factor | bevacizumab | vascular endothelial growth |
| | receptor | | factor A |
| doxorubicin, EDV | epidermal growth factor | pritumumab | vimentin |
| nanocells, | receptor | | |
| EnGenelC | | | |
| pan-HER | epidermal growth factor | pexidartinib | v-kit Hardy-Zuckerman 4 |
| | receptor | | feline sarcoma viral oncogene |
| | erb-b2 receptor tyrosine | | homologue |
| | kinase 2 | | colony stimulating factor 1 |
| | erb-b2 receptor tyrosine | | receptor |
| | kinase 3 | | fms-related tyrosine kinase 3 |
| trastuzumab | erb-b2 receptor tyrosine | galinpepimut-S | Wilms tumour 1 |
| deruxtecan | kinase 2 | | |
| trastuzumab | erb-b2 receptor tyrosine | adegramotide/nelati | Wilms tumour 1 |
| emtansine | kinase 2 | motide | |
| (vic-)trastuzumab | erb-b2 receptor tyrosine | JTCRO16 | Wilms tumour 1 |
| duocarmazine | kinase 2 | | |
| nelipepimut-S | erb-b2 receptor tyrosine | levamisole | Unknown |
| | kinase 2 | | |
| trastuzumab | erb-b2 receptor tyrosine | ladiratuzumab | Unknown |
| biosimilar, Merck | kinase 2 | vedotin | |
| & Co./Samsung | | | |
| Bioepis | | | |
| trastuzumab | erb-b2 receptor tyrosine | NSC-631570 | Unknown |
| biosimilar, | kinase 2 | | |
| Celltrion | | | |
| trastuzumab | erb-b2 receptor tyrosine | LN-145 | Unknown |
| biosimilar, Biocon | kinase 2 | | |
| trastuzumab | erb-b2 receptor tyrosine | INO-5401 | Unknown |
| biosimilar, | kinase 2 | | |
| Allergan/Amgen | | | |
| trastuzumab | erb-b2 receptor tyrosine | ANO1, Anson | Unknown |
| biosimilar, Pfizer | kinase 2 | Pharma | |
| AU101, Aurora | erb-b2 receptor tyrosine | GALE-302 | Unknown |
| Biopharma | kinase 2 | | |
| AU105, Aurora | erb-b2 receptor tyrosine | MAGE-A3 TCR, | Unknown |
| BioPharma | kinase 2 | Adaptimmune | |
| AE37 | erb-b2 receptor tyrosine | BTH-1677 | Unknown |
| | kinase 2 | | |
| trastuzumab | erb-b2 receptor tyrosine | lentinan | Unknown |
| | kinase 2 | | |
| pertuzumab | erb-b2 receptor tyrosine | Polysaccharide-K | Unknown |
| | kinase 2 | | |
| margetuximab | erb-b2 receptor tyrosine | Tice BCG, Organon | Unknown |
| | kinase 2 | | |
| ADXS31-164 | erb-b2 receptor tyrosine | IGEM-F | Unknown |
| | kinase 2 | | |
| ETBX-021 | erb-b2 receptor tyrosine | PV-10, Provectus | Unknown |
| | kinase 2 | | |
| seribantumab | erb-b2 receptor tyrosine | vitespen | Unknown |
| | kinase 3 | | |
| patritumab | erb-b2 receptor tyrosine | mifamurtide | Unknown |
| | kinase 3 | | |
| CDX-3379 | erb-b2 receptor tyrosine | melanoma vaccine, | Unknown |
| | kinase 3 | GSK | |
| elgemtumab | erb-b2 receptor tyrosine | Bacille Calmette- | Unknown |
| | kinase 3 | Guerin vaccine, ID | |
| | | Biomedical | |
| moxetumomab | eukaryotic translation | seviprotimut-l | Unknown |
| pasudotox | elongation factor 2 | | |
| | CD22 molecule | | |
| denileukin diftitox | eukaryotic translation | in situ autologous | Unknown |
| | elongation factor 2 | cancer vaccine, | |
| | interleukin 2 receptor, alpha | Immunophotonics | |
| MDNA55 | eukaryotic translation | IMA901 | Unknown |
| | elongation factor 2 | | |
| | interleukin 4 receptor | | |
| bemarituzumab | fibroblast growth factor | adagloxad | Unknown |
| | receptor 2 | simolenin | |
| DCVax-prostate, | folate hydrolase (prostate- | PVX-410 | Unknown |
| Northwest | specific membrane antigen) | | |
| Biotherapeutics | 1 | | |
| 177Lu-J591 | folate hydrolase (prostate- | viagenpumatucel-L | Unknown |
| | specific membrane antigen) | | |
| | 1 | | |
| tuberculosis | folate hydrolase (prostate- | GALE-301 | Unknown |
| vaccine (Mw), | specific membrane antigen) | | |
| Cadila; Cadi-05 | 1 | | |
| mirvetuximab | folate receptor 1 (adult) | EP-302, EpiThany | Unknown |
| soravtansine | | | |
| TPIV200 | folate receptor 1 (adult) | Bl 1361849 | Unknown |
| farletuzumab | folate receptor 1 (adult) | DPV-001 | Unknown |
| IGEM-FR | folate receptor 1 (adult) | Bacille Calmette- | Unknown |
| | | Guerin vaccine, | |
| | | Sanofi | |
| G17DT | gastrin | LAMP-Vax + pp65 | Unknown |
| | | DC, Immunomic | |
| | | Therapeutics | |
| codrituzumab | glypican 3 | NKG2D-CAR | Unknown |
| EP-100, | gonadotropin-releasing | BPX-501 | Unknown |
| EpiThany | hormone 1 (luteinizing- | | |
| | releasing hormone) | | |
| | luteinizing | | |
| | hormone/choriogonadotropi | | |
| | n receptor | | |
| naxitamab | growth differentiation factor | NK-92 cells | Unknown |
| | 2 | | |
| CDX-014 | hepatitis A virus cellular | LN-144 | Unknown |
| | receptor 1 | | |
| MBG453 | hepatitis A virus cellular | CLBS-23 | Unknown |
| | receptor 2 | | |
| histamine | histamine receptor H2 | DCVax-Direct, | Unknown |
| dihydrochloride | | Northwest | |
| | | Biotherapeutics | |
| entinostat | histone deacetylase 1 | melanoma vaccine, | Unknown |
| | | AVAX | |
| indoximod | indoleamine-pyrrole 2,3 | stapuldencel-T | Unknown |
| | dioxygenase | | |
| epacadostat | indoleamine-pyrrole 2,3 | dendritic cancer | Unknown |
| | dioxygenase | vaccine, DanDrit | |
| | | Biotech | |
| BMS-986205 | indoleamine-pyrrole 2,3 | DCVax-Brain | Unknown |
| | dioxygenase | brain cancer | |
| | | vaccine, Northwest | |
| | | Biotherapeutics | |
| JTX-2011 | inducible T-cell co- | tumor lysate | Unknown |
| | stimulator | particle-loaded | |
| | | dendritic cell | |
| | | vaccine, Perseus | |
| BMS-986226 | inducible T-cell co- | ERC1671 | Unknown |
| | stimulator | | |
| ADC W0101 | insulin-like growth factor 1 | BSK01 | Unknown |
| | receptor | TAPA pulsed DC | |
| | | vaccine | |
| ganitumab | insulin-like growth factor 1 | Oncoquest-CLL | Unknown |
| | receptor | vaccine | |
| istiratumab | insulin-like growth factor 1 | rocapuldencel-T | Unknown |
| | receptor | | |
| | erb-b2 receptor tyrosine | | |
| | kinase 3 | | |
| dusigitumab | insulin-like growth factor 1 | ATIR-101 | Unknown |
| | receptor | | |
| | insulin-like growth factor 2 | | |
| | receptor | | |
| EP-201, | insulin-like growth factor | TVI-Kidney-1 | Unknown |
| EpiThany | binding protein 2, 36kDa | | |
| citoplurikin | interferon gamma receptor | TVAX cancer | Unknown |
| | 1 | vaccine, TVAX | |
| | tumour necrosis factor | Biomedical | |
| | receptor superfamily, | | |
| | member 1A | | |
| MABp1 | interleukin 1, alpha | atezolizumab, | Unknown |
| | | companion | |
| | | diagnostic | |
| pegilodecakin | interleukin 10 | tumour infiltrating | Unknown |
| | | lymphocytes, | |
| | | I ovance | |
| | | Biotherapeutics-2 | |
| Ad-RTS-hlL-12 + | interleukin 12 receptor, | MAGE A-10TCR, | Unknown |
| veledimex | beta 1 | Adaptimmune | |
| tavokinogene | interleukin 12 receptor, | IMA101 | Unknown |
| telsaplasmid | beta 1 | | |
| | interleukin 12 receptor, | | |
| | beta 2 | | |
| EGEN-001 | interleukin 12A (natural | algenpantucel-L | Unknown |
| | killer cell stimulatory factor | | |
| | 1, cytotoxic lymphocyte | | |
| | maturation factor 1, p35) | | |
| | interleukin 12B (natural | | |
| | killer cell stimulatory factor | | |
| | 2, cytotoxic lymphocyte | | |
| | maturation factor 2, p40) | | |
| SL-701 | interleukin 13 receptor, | Tumor Necrosis | Unknown |
| | alpha 2 | Therapy, Peregrine | |
| | EPH receptor A2 | | |
| | baculoviral IAP repeat | | |
| | containing 5 | | |
| ALT-803 | interleukin 15 receptor, | imiquimod | Unknown |
| | alpha | | |
| Multikine, Cel-Sci | interleukin 2 receptor, alpha | LOAd703 | Unknown |
| ALT-801 | interleukin 2 receptor, alpha | CG0070 | Unknown |
| high-affinity | interleukin 2 receptor, alpha | dinutuximab | Unknown |
| Natural Killer | | | |
| (haNK) cells, | | | |
| NantKwest | | | |
| interleukin-2, | interleukin 2 receptor, alpha | bavituximab | Unknown |
| Roche | | | |
| aldesleukin | interleukin 2 receptor, alpha | ensituximab | Unknown |
| NKTR-214 | interleukin 2 receptor, beta | pidilizumab | Unknown |
| talacotuzumab | interleukin 3 receptor, alpha | BMS-986218 | Unknown |
| | (low affinity) | | |
| SL-401 | interleukin 3 receptor, alpha | BMS-986012 | Unknown |
| | (low affinity) | | |
| siltuximab | interleukin 6 (interferon, | ADXS31-142 | Unknown |
| | beta 2) | | |
| HuMax-IL8 | interleukin 8 | GI-6301 | Unknown |
| PSA/IL-2/GM- | kallikrein-related peptidase | GI-4000 | Unknown |
| CSF | 3 | | |
| rilimogene | kallikrein-related peptidase | JNJ-64041757 | Unknown |
| galvacirepvec | 3 | | |
| | CD80 molecule | | |
| | intercellular adhesion | | |
| | molecule 1 | | |
| | CD58 molecule | | |
| monalizumab | killer cell lectin-like receptor | HPV vaccine | Unknown |
| | subfamily C, member 1 | (Cervarix), GSK | |
| ramucirumab | kinase insert domain | HPV vaccine | Unknown |
| | receptor | (Gardasil), CSL | |
| ubenimex | leucotriene A4 hydrolase | Sym015 | Unknown |
| | leucotriene B4 receptor | | |
| IMP321 | lymphocyte-activation gene | diphenylcycloprope | Unknown |
| | 3 | none | |
| LAG525 | lymphocyte-activation gene | ISA101 | Unknown |
| | 3 | | |
| relatlimab | lymphocyte-activation gene | | |
| | 3 | | |

TABLE 4
Sequences

| Name | | | |
|---|---|---|---|
| (Target, if | | SEQ | |
| applicable) | Region | ID | Sequence |
| JTX-2011 | Heavy | 1 | EVQLVESGGGLVQPGGSLRLSCAASGFTFSDYWMDWVRQAPGKGLVWVSNI |
| (ICOS) | Chain | | DEDGSITEYSPFVKGRFTISRDNAKNTLYLQMNSLRAEDTAVYYCTRWGRF |
| | | | GFDSWGQGTLVTVSSASTKGPSVFPLAPSSKSTSGGTAALGCLVKDYFPEP |
| | | | VTVSWNSGALTSGVHTFPAVLQSSGLYSLSSVVTVPSSSLGTQTYICNVNH |
| | | | KPSNTKVDKKVEPKSCDKTHTCPPCPAPELLGGPSVFLFPPKPKDTLMISR |
| | | | TPEVTCVVVDVSHEDPEVKFNWYVDGVEVHNAKTKPREEQYNSTYRVVSVL |
| | | | TVLHQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPG |
| JTX-2011 | Light | 2 | DIVMTQSPDSLAVSLGERATINCKSSQSLLSGSFNYLTWYQQKPGQ |
| (ICOS) | Chain | | PPKLLIFYASTRHTGVPDRFSGSGSGTDFTLTISSLQAEDVAVYYC |
| | | | HHHYNAPPTFGPGTKVDIKRTVAAPSVFIFPPSDEQLKSGTASVVC |
| | | | LLNNFYPREAKVQWKVDNALQSGNSQESVTEQDSKDSTYSLSSTLT |
| | | | LSKADYEKHKVYACEVTHQGLSSPVTKSFNRGEC |
| JTX-2011 | Heavy | 3 | EVQLVESGGGLVQPGGSLRLSCAASGFTFSDYWMDWVRQAPGKGLVWVSNI |
| (ICOS) | Chain | | DEDGSITEYSPFVKGRFTISRDNAKNTLYLQMNSLRAEDTAVYYCTRWGRF |
| | Variable | | GFDSWGQGTLVTVSS |
| | Region | | |
| JTX-2011 | Light | 4 | DIVMTQSPDSLAVSLGERATINCKSSQSLLSGSFNYLTWYQQKPGQPPKLL |
| (ICOS) | Chain | | IFYASTRHTGVPDRFSGSGSGTDFTLTISSLQAEDVAVYYCHHHYNAPPTF |
| | Variable | | GPGTKVDIK |
| | Region | | |
| JTX-2011 | HCDR1 | 5 | GFTFSDYWMD |
| (ICOS) | | | |
| JTX-2011 | HCDR2 | 6 | NIDEDGSITEYSPFVK |
| (ICOS) | | | |
| JTX-2011 | HCDR3 | 7 | WGRFGFDS |
| (ICOS) | | | |
| JTX-2011 | LCDR1 | 8 | KSSQSLLSGSFNYLT |
| (ICOS) | | | |
| JTX-2011 | LCDR2 | 9 | YASTRHT |
| (ICOS) | | | |
| JTX-2011 | LCDR3 | 10 | HHHYNAPPT |
| (ICOS) | | | |
| Human ICOS | | 11 | MKSGLWYFFLFCLRIKVLTGEINGSANYEMFIFHNGGVQILCKYPDIVQQF |
| precursor | | | KMQLLKGGQILCDLTKTKGSGNTVSIKSLKFCHSQLSNNSVSFFLYNLDHS |
| with signal | | | HANYYFCNLSIFDPPPFKVTLTGGYLHIYESQLCCQLKFWLPIGCAAFVVV |
| sequence | | | CILGCILICWLTKKKYSSSVHDPNGEYMFMRAVNTAKKSRLTDVTL |
| | | | (Intra-cellar Region is under-lined) |
| Human ICOS, | | 12 | EINGSANYEMFIFHNGGVQILCKYPDIVQQFKMQLLKGGQILCDLTKTKGS |
| mature | | | GNTVSIKSLKFCHSQLSNNSVSFFLYNLDHSHANYYFCNLSIFDPPPFKVT |
| | | | LTGGYLHIYESQLCCQLKFWLPIGCAAFVVVCILGCILICWLTKKKYSSSV |
| | | | HDPNGEYMFMRAVNTAKKSRLTDVTL |
| | | | (Intra-cellar Region is under-lined) |
| Mouse (Mus | | 13 | MKPYFCRVFVFCFLIRLLTGEINGSADHRMFSFHNGGVQISCKYPE |
| musculus) | | | TVQQLKMRLFREREVLCELTKTKGSGNAVSIKNPMLCLYHLSNNSV |
| ICOS | | | SFFLNNPDSSQGSYYFCSLSIFDPPPFQ |
| precursor | | | ERNLSGGYLHIYESQLCCQLKLWLPVGCAAFVVVLLFGCILIIWFS |
| | | | KKKYGSSVHDPNSEYMFMAAVNTNKKSRLAGVTS |
| Mouse (Mus | | 14 | EINGSADHRMFSFHNGGVQISCKYPETVQQLKMRLFREREVLCELT |
| musculus) | | | KTKGSGNAVSIKNPMLCLYHLSNNSVSFFLNNPDSSQGSYYFCSLS |
| ICOS, mature | | | IFDPPPFQERNLSGGYLHIYESQLCCQLKLWLPVGCAAFVVVLLFG |
| | | | CILIIWFSKKKYGSSVHDPNSEYMFMAAVNTNKKSRLAGVTS |
| Rat (Rattus | | 15 | MKPYFSCVFVFCFLIKLLTGELNDLANHRMFSFHDGGVQISCNYPE |
| norvegicus) | | | TVQQLKMQLFKDREVLCDLTKTKGSGNTVSIKNPMSCPYQLSNNSV |
| ICOS | | | SFFLDNADSSQGSYFLCSLSIFDPPPFQEKNLSGGYLLIYESQLCC |
| precursor | | | QLKLWLPVGCAAFVAALLFGCIFIVWFAKKKYRSSVHDPNSEYMFM |
| | | | AAVNTNKKSRLAGMTS |
| Rat (Rattus | | 16 | ELNDLANHRMFSFHDGGVQISCNYPETVQQLKMQLFKDREVLCDLTKTKGS |
| norvegicus) | | | GNTVSIKNPMSCPYQLSNNSVSFFLDNADSSQGSYFLCSLSIFDPPPFQEK |
| ICOS, mature | | | NLSGGYLLIYESQLCCQLKLWLPVGCAAFVAALLFGCIFIVWFAKKKYRSS |
| | | | VHDPNSEYMFMAAVNTNKKSRLAGMTS |
| Cynomolgus | | 17 | MKSGLWYFFLFCLHMKVLTGEINGSANYEMFIFHNGGVQILCKYPDIVQQF |
| monkey | | | KMQLLKGGQILCDLTKTKGSGNKVSIKSLKFCHSQLSNNSVSFFLYNLDRS |
| (Macaca | | | HANYYFCNLSIFDPPPFKVTLTGGYLHIYESQLCCQLKFWLPIGCATFVVV |
| fascicularis) | | | CIFGCILICWLTKKKYSSTVHDPNGEYMFMRAVNTAKKSRLTGTTP |
| ICOS, | | | |
| precursor | | | |
| Cynomolgus | | 18 | EINGSANYEMFIFHNGGVQILCKYPDIVQQFKMQLLKGGQILCDLT |
| monkey | | | KTKGSGNKVSIKSLKFCHSQLSNNSVSFFLYNLDRSHANYYFCNLS |
| (Macaca | | | IFDPPPFKVTLTGGYLHIYESQLCCQLKFWLPIGCATFVVVCIFGC |
| fascicularis) | | | ILICWLTKKKYSSTVHDPNGEYMFMRAVNTAKKSRLTGTTP |
| ICOS, mature | | | |
| JNC-1 (PD-1) | Heavy | 19 | QVQLVQSGAEVKKPGASVKVSCKASGYTFPSYYMHWVRQAPGQGLEWMGII |
| | Chain | | NPEGGSTAYAQKFQGRVTMTRDTSTSTVYMELSSLRSEDTAVYYCARGGTY |
| | Variable | | YDYTYWGQGTLVTVSS |
| | Region | | |
| JNC-1 (PD-1) | HCDR1 | 20 | YTFPSYYMH |
| JNC-1 (PD-1) | HCDR2 | 21 | IINPEGGSTAYAQKFQG |
| JNC-1 (PD-1) | HCDR3 | 22 | ARGGTYYDYTY |
| JNC-1 (PD-1) | Light | 23 | DIQMTQSPSTLSASVGDRVTITCRASQSISSWLAWYQQKPGKAPKLLIYEA |
| | Chain | | SSLESGVPSRFSGSGSGTEFTLTISSLQPDDFATYYCQQYNSFPPTFGGGT |
| | Variable | | KVEIK |
| | Region | | |
| JNC-1 (PD-1) | LCDR1 | 24 | RASQSISSWLA |
| JNC-1 (PD-1) | LCDR2 | 25 | EASSLES |
| JNC-1 (PD-1) | LCDR3 | 26 | QQYNSFPPT |
| 2M13 (ICOS | Heavy | 27 | EVQLQQSGAELVRPGAVVKLSCKASGFDIKDYYMHWVQQRPEQGLEWIGWI |
| intra- | Chain | | DPENGNAVYDPQFQGKASITADTSSNTAYLQLSSLTSEDTAVYYCASDYYG |
| cellular) | Variable | | SKGYLDVWGAGTTVTVSS |
| | Region | | |
| 2M13 (ICOS | HCDR1 | 28 | DYYMH |
| intra- | | | |
| cellular) | | | |
| 2M13 (ICOS | HCDR2 | 29 | WIDPENGNAVYDPQFQG |
| intra- | | | |
| cellular) | | | |
| 2M13 (ICOS | HCDR3 | 30 | DYYGSKGYLDV |
| intra- | | | |
| cellular) | | | |
| 2M13 (ICOS | Light | 31 | QIVLTQSPTIMSASPGEKVTITCSASSSVSYMHWFQQKPGTSPKLWIYSTS |
| intra- | Chain | | NLASGVPARFGGSRSGTSYSLTISRMEAEDAATYYCQQRSSYPFTFGSGTK |
| cellular) | Variable | | LEIK |
| | Region | | |
| 2M13 (ICOS | LCDR1 | 32 | SASSSVSYMH |
| intra- | | | |
| cellular) | | | |
| 2M13 (ICOS | LCDR2 | 33 | STSNLAS |
| intra- | | | |
| cellular) | | | |
| 2M13 (ICOS | LCDR3 | 34 | QQRSSYPFT |
| intra- | | | |
| cellular) | | | |
| 2M19 (ICOS | Heavy | 35 | EVQLQQSGAELVRSGASVKLSCTTSAFNIIDYYMHWVIQRPEQGLEWIAWI |
| intra- | Chain | | DPENGDPEYAPKFQDKATMTTDTSSNTAYLQLSSLTSEDTAVYYCTAWRGF |
| cellular) | Variable | | AYWGQGTLVTVSA |
| | Region | | |
| 2M19 (ICOS | HCDR1 | 36 | DYYMH |
| intra- | | | |
| cellular) | | | |
| 2M19 (ICOS | HCDR2 | 37 | WIDPENGDPEYAPKFQD |
| intra- | | | |
| cellular) | | | |
| 2M19 (ICOS | HCDR3 | 38 | WRGFAY |
| intra- | | | |
| cellular) | | | |
| 2M19 (ICOS | Light | 39 | DVVMTQTPLSLPVSLGDQASISCRSSQSLVHSNGNTYLHWYLQKPGQSPKL |
| intra- | Chain | | LIYKVSNRFSGVPDRFSGSGSGTDFTLKISRVEAEDLGVYFCSQSIHVPPT |
| cellular) | Variable | | FGGGTKLEIK |
| | Region | | |
| 2M19 (ICOS | LCDR1 | 40 | RSSQSLVHSNGNTYLH |
| intra- | | | |
| cellular) | | | |
| 2M19 (ICOS | LCDR2 | 41 | KVSNRFS |
| intra- | | | |
| cellular) | | | |
| 2M19 (ICOS | LCDR3 | 42 | SQSIHVPPT |
| intra- | | | |
| cellular) | | | |
| JTX-4014 | Heavy | 43 | QVQLVQSGAEVKKPGASVKVSCKASGYTFPSYYMHWVRQAPGQGLEWMGII |
| (PD-1) | Chain | | NPEGGSTAYAQKFQGRVTMTRDTSTSTVYMELSSLRSEDTAVYYCARGGTY |
| | Variable | | YDYTYWGQGTLVTVSS |
| | Region | | |
| JTX-4014 | HCDR1 | 44 | YTFPSYYMH |
| (PD-1) | | | |
| JTX-4014 | HCDR2 | 45 | IINPEGGSTAYAQKFQG |
| (PD-1) | | | |
| JTX-4014 | HCDR3 | 46 | ARGGTYYDYTY |
| (PD-1) | | | |
| JTX-4014 | Heavy | 47 | DIQMTQSPSTLSASVGDRVTITCRASQSISSWLAWYQQKPGKAPKLLIYEA |
| (PD-1) | Chain | | SSLESGVPSRFSGSGSGTEFTLTISSLQPDDFATYYCQQYNSFPPTFGGGT |
| | Variable | | KVEIK |
| | Region | | |
| JTX-4014 | LCDR1 | 48 | RASQSISSWLA |
| (PD-1) | | | |
| JTX-4014 | LCDR2 | 49 | EASSLES |
| (PD-1) | | | |
| JTX-4014 | LCDR3 | 50 | QQYNSFPPT |
| (PD-1) | | | |
| JTX-4014 | Heavy | 51 | QVQLVQSGAEVKKPGASVKVSCKASGYTFPSYYMHWVRQAPGQGLEWMGII |
| (PD-1) | Chain | | NPEGGSTAYAQKFQGRVTMTRDTSTSTVYMELSSLRSEDTAVYYCARGGTY |
| | | | YDYTYWGQGTLVTVSSASTKGPSVFPLAPCSRSTSESTAALGCLVKDYFPE |
| | | | PVTVSWNSGALTSGVHTFPAVLQSSGLYSLSSVVTVPSSSLGTKTYTCNVD |
| | | | HKPSNTKVDKRVESKYGPPCPPCPAPEFLGGPSVFLFPPKPKDTLMISRTP |
| | | | EVTCVVVDVSQEDPEVQFNWYVDGVEVHNAKTKPREEQFNSTYRVVSVLTV |
| | | | LHQDWLNGKEYKCKVSNKGLPSSIEKTISKAKGQPREPQVYTLPPSQEEMT |
| | | | KNQVSLTCLVKGFYPSDIAVEWESNGQPENNYKTTPPVLDSDGSFFLYSRL |
| | | | TVDKSRWQEGNVFSCSVMHEALHNHYTQKSLSLSLGK |
| JTX-4014 | Light | 52 | DIQMTQSPSTLSASVGDRVTITCRASQSISSWLAWYQQKPGKAPKLLIYEA |
| (PD-1) | Chain | | SSLESGVPSRFSGSGSGTEFTLTISSLQPDDFATYYCQQYNSFPPTFGGGT |
| | | | KVEIKRTVAAPSVFIFPPSDEQLKSGTASVVCLLNNFYPREAKVQWKVDNA |
| | | | LQSGNSQESVTEQDSKDSTYSLSSTLTLSKADYEKHKVYACEVTHQGLSSP |
| | | | VTKSFNRGEC |

What is claimed is:

1. A method for treating a subject having cancer characterized by an elevated RNA signature score, the method comprising administering to the subject an ICOS agonist and a PD1 antagonist.

2. A method for identifying a subject whose cancer is likely to respond to combination ICOS agonist and PD1 antagonist therapy, the method comprising determining the RNA signature score of a sample of the cancer of the subject, wherein detection of an elevated RNA signature score in the sample indicates that the subject is likely to respond to the combination therapy.

3. A method of selecting a cancer therapy for a subject having cancer, the method comprising determining the RNA signature score of a sample of the cancer of the subject, wherein detection of an elevated RNA signature score in the sample indicates selection of a combination of ICOS agonist and PD1 antagonist therapy for the subject.

4. A method of selecting a subject having cancer for combination ICOS agonist and PD1 antagonist therapy, the method comprising determining the RNA signature score of a sample of the cancer of the subject, wherein detection of an elevated RNA signature score in the sample indicates selection of a subject for the combination therapy.

5. A method of determining whether a subject having cancer may develop an ICOShi CD4+ T cell population, the method comprising determining the RNA signature score of a sample of the cancer of the subject, wherein detection of an elevated RNA signature score in the sample indicates that the subject may develop an ICOShi CD4+ T cell population.

6. The method of any one of claims 2 to 5, further comprising administering an ICOS agonist and a PD-1 antagonist to the subject.

7. A method for increasing the length of duration of response to a PD1 antagonist in a subject having cancer, the method comprising administering an ICOS agonist to the subject, wherein the cancer of the subject has an elevated RNA signature score.

8. The method of any one of claims 1 to 7, wherein a subject having cancer with an elevated RNA signature score has improved tumor regression, duration of response, or RECIST criteria when treated with a combination of an ICOS agonist and a PD1 antagonist.

9. The method of claim 8, wherein the subject has improved overall response rate, progression free survival, stable disease, or overall survival.

10. The method of any one of claims 1 to 9, wherein the subject has an increased level of ICOShi CD4+ T cells.

11. The method of any one of claims 1 and 7 to 10, further comprising determining the RNA signature score of a sample of the cancer from the subject.

12. The method of any one of claims 2 to 6 and 11, wherein the RNA signature score is determined by evaluation of RNA levels of the components of the RNA signature.

13. The method of claim 12, wherein the RNA signature score is determined by nanostring technology.

14. The method of any one of claims 2 to 11, wherein determining of the RNA signature score comprises detection of the levels of each RNA listed in Table 1, normalizing the levels of RNA listed in Table 1 against the levels of standards in Table 2, and weighting the normalized levels using the fourth column of Table 1.

15. The method of any one of claims 1 to 14, wherein the RNA signature score is elevated if it is measured to be about 2.97 or above.

16. The method of any one of claims 1 to 14, wherein the elevated RNA signature score is about 3.39 or above.

17. The method of any one of claims 1 to 14, wherein the elevated RNA signature score is about 3.40 or above.

18. The method of any one of claims 1 to 14, wherein the elevated RNA signature score is 3.40.

19. The method of any one of claims 1 to 14, wherein the elevated RNA signature score is about 3.58 or above.

20. The method of any one of claims 1 to 14, wherein the elevated RNA signature score is between about 2.97 and about 3.58.

21. The method of any one of claims 1 to 20, wherein the ICOS agonist is an antibody.

22. The method of claim 21, wherein the ICOS agonist antibody comprises (a) a heavy chain comprising the amino acid sequence of SEQ ID NO: 1, or (b) a light chain comprising the amino acid sequence of SEQ ID NO: 2.

23. The method of claim 22, wherein the anti-ICOS antibody agonist comprises (a) a heavy chain comprising the amino acid sequence of SEQ ID NO: 1, and (b) a light chain comprising the amino acid sequence of SEQ ID NO: 2.

24. The method of claim 21, wherein the ICOS agonist antibody is selected from the group consisting of JTX-2011, BMS-986226, and GSK3359609.

25. The method of any one of claims 1 to 24, wherein the PD1 antagonist is directed against PD1.

26. The method of any one of claims 1 to 24, wherein the PD1 antagonist is directed against PD-L1.

27. The method of any one of claims 1 to 26, wherein the PD1 antagonist is an antibody.

28. The method of claim 27, wherein the PD1 antagonist antibody is selected from the group consisting of: JTX-4014, nivolumab, pidilizumab, lambrolizumab, pembrolizumab, cemiplimab, avelumab, atezolizumab tislelizumab, durvalumab, spartalizumab, genolimzumab, BGB-A317, RG-7446, AMP-224, BMS-936559, AMP-514, MDX-1105, AB-011, RG7446/MPDL3280A, KD-033, AGEN-2034, STI-A1010, STI-A1110, TSR-042, CX-072, JNJ-63723283, and JNC-1.

29. The method of claim 26, wherein the PD1 antagonist antibody is JTX-4014.

30. The method of any one of claims 1 to 29, wherein the cancer of the subject is selected from the group consisting of gastric cancer, breast cancer, which optionally is triple negative breast cancer (TNBC), non-small cell lung cancer (NSCLC), melanoma, renal cell carcinoma (RCC), bladder cancer, endometrial cancer, diffuse large B-cell lymphoma (DLBCL), Hodgkin's lymphoma, ovarian cancer, head and neck squamous cell cancer (HNSCC), anal cancer, biliary cancer, colorectal cancer, and esophageal cancer.

31. A kit for use in determining whether to administer a combination of ICOS agonist and PD-1 antagonist therapy to a subject having cancer, the kit comprising primers and/or probes for detecting the components of an RNA signature as described herein in a sample of the cancer of the subject.

32. An ICOS agonist and a PD1 antagonist for use in the treatment of a subject having cancer characterized by an elevated RNA signature score.

33. The ICOS agonist and PD1 antagonist for use according to claim 32, wherein the subject having cancer characterized by an elevated RNA signature score has:
(a) improved tumor regression, duration of response, or RECIST criteria when treated with a combination of an ICOS agonist and a PD1 antagonist;
(b) improved overall response rate, progression free survival, stable disease, or overall survival; or
(c) an increased level of ICOShi CD4+ T cells.

34. The ICOS agonist and PD1 antagonist for use according to claim 32 or 33, wherein the RNA signature score of a sample of the cancer from the subject is determined, wherein:
(a) the RNA signature score is determined by evaluation of RNA levels of the components of the RNA signature;
(b) the RNA signature score is determined by nanostring technology; or
(c) determining of the RNA signature score comprises detection of the levels of each RNA listed in Table 1, normalizing the levels of RNA listed in Table 1 against the levels of standards in Table 2, and weighting the normalized levels using the fourth column of Table 1.

35. The ICOS agonist and PD1 antagonist for use according to any one of claims 32 to 34, wherein;
(a) the RNA signature score is elevated if it is measured to be about 2.97 or above;
(b) the elevated RNA signature score is about 3.39 or above;
(c) the elevated RNA signature score is about 3.40 or above;
(d) the elevated RNA signature score is 3.40;
(e) the elevated RNA signature score is about 3.58 or above; or
(f) the elevated RNA signature score is between about 2.97 and about 3.58.

36. The ICOS agonist and PD1 antagonist for use according to any one of claims 32 to 35, wherein:
(a) the ICOS agonist is an antibody;
(b) the ICOS agonist antibody comprises (i) a heavy chain comprising the amino acid sequence of SEQ ID NO: 1, or (ii) a light chain comprising the amino acid sequence of SEQ ID NO: 2;
(c) the anti-ICOS antibody agonist comprises (i) a heavy chain comprising the amino acid sequence of SEQ ID NO: 1, and (ii) a light chain comprising the amino acid sequence of SEQ ID NO: 2; or
(d) the ICOS agonist antibody is selected from the group consisting of JTX-2011, BMS-986226, and GSK3359609.

37. The ICOS agonist and PD1 antagonist for use according to any one of claims 32 to 36, wherein:
(a) the PD1 antagonist is directed against PD1;
(b) the PD1 antagonist is directed against PD-L1;
(c) the PD1 antagonist is an antibody;
(d) the PD1 antagonist antibody is selected from the group consisting of: JTX-4014, nivolumab, pidilizumab, lambrolizumab, pembrolizumab, cemiplimab, avelumab, atezolizumab tislelizumab, durvalumab, spartalizumab, genolimzumab, BGB-A317, RG-7446, AMP-224, BMS-936559, AMP-514, MDX-1105, AB-011, RG7446/MPDL3280A, KD-033, AGEN-2034, STI-A1010, STI-A1110, TSR-042, CX-072, JNJ-63723283, and JNC-1; or
(e) the PD1 antagonist antibody is JTX-4014.

38. The ICOS agonist and PD1 antagonist for use according to any one of claims 32 to 37, wherein the cancer of the subject is selected from the group consisting of gastric cancer, breast cancer, which optionally is triple negative breast cancer (TNBC), non-small cell lung cancer (NSCLC), melanoma, renal cell carcinoma (RCC), bladder cancer, endometrial cancer, diffuse large B-cell lymphoma (DLBCL), Hodgkin's lymphoma, ovarian cancer, head and neck squamous cell cancer (HNSCC), anal cancer, biliary cancer, colorectal cancer, and esophageal cancer.

39. An ICOS agonist for use in increasing the length of duration of response to a PD1 antagonist in a subject having cancer characterized by an elevated RNA signature score.

40. The ICOS agonist for use according to claim 39, wherein the subject having cancer characterized by an elevated RNA signature score has:
(a) improved tumor regression, duration of response, or RECIST criteria when treated with a combination of an ICOS agonist and a PD1 antagonist;
(b) improved overall response rate, progression free survival, stable disease, or overall survival; or
(c) an increased level of ICOShi CD4+ T cells.

41. The ICOS agonist for use according to claim 39 or 40, wherein the RNA signature score of a sample of the cancer from the subject is determined, wherein:
(a) the RNA signature score is determined by evaluation of RNA levels of the components of the RNA signature;
(b) the RNA signature score is determined by nanostring technology; or
(c) determining of the RNA signature score comprises detection of the levels of each RNA listed in Table 1, normalizing the levels of RNA listed in Table 1 against the levels of standards in Table 2, and weighting the normalized levels using the fourth column of Table 1.

42. The ICOS agonist for use according to any one of claims 39 to 41, wherein;
(a) the RNA signature score is elevated if it is measured to be about 2.97 or above;
(b) the elevated RNA signature score is about 3.39 or above;
(c) the elevated RNA signature score is about 3.40 or above;
(d) the elevated RNA signature score is 3.40;
(e) the elevated RNA signature score is about 3.58 or above; or
(f) the elevated RNA signature score is between about 2.97 and about 3.58.

43. The ICOS agonist for use according to any one of claims 39 to 42, wherein:
(a) the ICOS agonist is an antibody;
(b) the ICOS agonist antibody comprises (i) a heavy chain comprising the amino acid sequence of SEQ ID NO: 1, or (ii) a light chain comprising the amino acid sequence of SEQ ID NO: 2;
(c) the anti-ICOS antibody agonist comprises (i) a heavy chain comprising the amino acid sequence of SEQ ID NO: 1, and (ii) a light chain comprising the amino acid sequence of SEQ ID NO: 2; or
(d) the ICOS agonist antibody is selected from the group consisting of JTX-2011, BMS-986226, and GSK3359609.

44. The ICOS agonist for use according to any one of claims 39 to 43, wherein:
(a) the PD1 antagonist is directed against PD1;
(b) the PD1 antagonist is directed against PD-L1;
(c) the PD1 antagonist is an antibody;
(d) the PD1 antagonist antibody is selected from the group consisting of: JTX-4014, nivolumab, pidilizumab, lambrolizumab, pembrolizumab, cerniplimab, avelumab, atezolizumab tislelizumab, durvalumab, spartalizumab, genolimzumab, BGB-A317, RG-7446, AMP-224, BMS-936559, AMP-514, MDX-1105, AB-011, RG7446/MPDL3280A, KD-033, AGEN-2034, STI-A1010, STI-A1110, TSR-042, CX-072, JNJ-63723283, and JNC-1; or
(e) the PD1 antagonist antibody is JTX-4014.

45. The ICOS agonist for, use according to any one of claims 39 to 44, wherein the cancer of the subject is selected from the group consisting of gastric cancer, breast cancer, which optionally is triple negative breast cancer (TNBC), non-small cell lung cancer (NSCLC), melanoma, renal cell carcinoma (RCC), bladder cancer, endometrial cancer, diffuse large B-cell lymphoma (DLBCL), Hodgkin's lymphoma, ovarian cancer, head and neck squamous cell cancer (HNSCC), anal cancer, biliary cancer, colorectal cancer, and esophageal cancer.


SEQUENCE LISTING (52 sequences)

SEQ ID NO: 1 - protein, 446 aa, Artificial Sequence
  Synthetic Construct
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 218 aa, Artificial Sequence
  Synthetic Construct
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 117 aa, Artificial Sequence
  Synthetic Construct
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 4 - protein, 111 aa, Artificial Sequence
  Synthetic Construct
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 5 - protein, 10 aa, Artificial Sequence
  Synthetic Construct
GFTFSDYWMD

SEQ ID NO: 6 - protein, 16 aa, Artificial Sequence
  Synthetic Construct
NIDEDGSITEYSPFVK

SEQ ID NO: 7 - protein, 8 aa, Artificial Sequence
  Synthetic Construct
WGRFGFDS

SEQ ID NO: 8 - protein, 15 aa, Artificial Sequence
  Synthetic Construct
KSSQSLLSGSFNYLT

SEQ ID NO: 9 - protein, 7 aa, Artificial Sequence
  Synthetic Construct
YASTRHT

SEQ ID NO: 10 - protein, 9 aa, Artificial Sequence
  Synthetic Construct
HHHYNAPPT

SEQ ID NO: 11 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 12 - protein, 179 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 13 - protein, 200 aa, Mus musculus
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 14 - protein, 180 aa, Mus musculus
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 15 - protein, 200 aa, Rattus norvegicus
MKPYFSCVFVFCFLIKLLTGELNDLANHRMFSFHDGGVQISCNYPETVQQLKMQLFKDREVLCDLTKTKGSGNTVSIKNPMSCPYQLSNNSVSFFLDNADSSQGSYFLCSLSIFDPPPFQEKNLSGGYLLIYESQLCCQLKLWLPVGCAAFVAALLFGCIFIVWFAKKKYRSSVHDPNSEYMFMAAVNTNKKSRLAGMTS

SEQ ID NO: 16 - protein, 180 aa, Rattus norvegicus
ELNDLANHRMFSFHDGGVQISCNYPETVQQLKMQLFKDREVLCDLTKTKGSGNTVSIKNPMSCPYQLSNNSVSFFLDNADSSQGSYFLCSLSIFDPPPFQEKNLSGGYLLIYESQLCCQLKLWLPVGCAAFVAALLFGCIFIVWFAKKKYRSSVHDPNSEYMFMAAVNTNKKSRLAGMTS

SEQ ID NO: 17 - protein, 199 aa, Macaca fascicularis
MKSGLWYFFLFCLHMKVLTGEINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 18 - protein, 179 aa, Macaca fascicularis
EINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 19 - protein, 118 aa, Artificial Sequence
  Synthetic Construct
QVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSS

SEQ ID NO: 20 - protein, 9 aa, Artificial Sequence
  Synthetic Construct
YTFPSYYMH

SEQ ID NO: 21 - protein, 17 aa, Artificial Sequence
  Synthetic Construct
IINPEGGSTAYAQKFQG

SEQ ID NO: 22 - protein, 11 aa, Artificial Sequence
  Synthetic Construct
ARGGTYYDYTY

SEQ ID NO: 23 - protein, 107 aa, Artificial Sequence
  Synthetic Construct
DIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIK

SEQ ID NO: 24 - protein, 11 aa, Artificial Sequence
  Synthetic Construct
RASQSISSWLA

SEQ ID NO: 25 - protein, 7 aa, Artificial Sequence
  Synthetic Construct
EASSLES

SEQ ID NO: 26 - protein, 9 aa, Artificial Sequence
  Synthetic Construct
QQYNSFPPT

SEQ ID NO: 27 - protein, 120 aa, Artificial Sequence
  Synthetic Construct
EVQLQQSGAELVRPGAVVKLSCKASGFDIKDYYMHWVQQRPEQGLEWIGWIDPENGNAVYDPQFQGKASITADTSSNTAYLQLSSLTSEDTAVYYCASDYYGSKGYLDVWGAGTTVTVSS

SEQ ID NO: 28 - protein, 5 aa, Artificial Sequence
  Synthetic Construct
DYYMH

SEQ ID NO: 29 - protein, 17 aa, Artificial Sequence
  Synthetic Construct
WIDPENGNAVYDPQFQG

SEQ ID NO: 30 - protein, 11 aa, Artificial Sequence
  Synthetic Construct
DYYGSKGYLDV

SEQ ID NO: 31 - protein, 106 aa, Artificial Sequence
  Synthetic Construct
QIVLTQSPTIMSASPGEKVTITCSASSSVSYMHWFQQKPGTSPKLWIYSTSNLASGVPARFGGSRSGTSYSLTISRMEAEDAATYYCQQRSSYPFTFGSGTKLEIK

SEQ ID NO: 32 - protein, 10 aa, Artificial Sequence
  Synthetic Construct
SASSSVSYMH

SEQ ID NO: 33 - protein, 7 aa, Artificial Sequence
  Synthetic Construct
STSNLAS

SEQ ID NO: 34 - protein, 9 aa, Artificial Sequence
  Synthetic Construct
QQRSSYPFT

SEQ ID NO: 35 - protein, 115 aa, Artificial Sequence
  Synthetic Construct
EVQLQQSGAELVRSGASVKLSCTTSAFNIIDYYMHWVIQRPEQGLEWIAWIDPENGDPEYAPKFQDKATMTTDTSSNTAYLQLSSLTSEDTAVYYCTAWRGFAYWGQGTLVTVSA

SEQ ID NO: 36 - protein, 5 aa, Artificial Sequence
  Synthetic Construct
DYYMH

SEQ ID NO: 37 - protein, 17 aa, Artificial Sequence
  Synthetic Construct
WIDPENGDPEYAPKFQD

SEQ ID NO: 38 - protein, 6 aa, Artificial Sequence
  Synthetic Construct
WRGFAY

SEQ ID NO: 39 - protein, 112 aa, Artificial Sequence
  Synthetic Construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSIHVPPTFGGGTKLEIK

SEQ ID NO: 40 - protein, 16 aa, Artificial Sequence
  Synthetic Construct
RSSQSLVHSNGNTYLH

SEQ ID NO: 41 - protein, 7 aa, Artificial Sequence
  Synthetic Construct
KVSNRFS

SEQ ID NO: 42 - protein, 9 aa, Artificial Sequence
  Synthetic Construct
SQSIHVPPT

SEQ ID NO: 43 - protein, 118 aa, Artificial Sequence; identical to SEQ ID NO: 19
  Synthetic Construct

SEQ ID NO: 44 - protein, 9 aa, Artificial Sequence
  Synthetic Construct
YTFPSYYMH

SEQ ID NO: 45 - protein, 17 aa, Artificial Sequence
  Synthetic Construct
IINPEGGSTAYAQKFQG

SEQ ID NO: 46 - protein, 11 aa, Artificial Sequence
  Synthetic Construct
ARGGTYYDYTY

SEQ ID NO: 47 - protein, 107 aa, Artificial Sequence; identical to SEQ ID NO: 23
  Synthetic Construct

SEQ ID NO: 48 - protein, 11 aa, Artificial Sequence
  Synthetic Construct
RASQSISSWLA

SEQ ID NO: 49 - protein, 7 aa, Artificial Sequence
  Synthetic Construct
EASSLES

SEQ ID NO: 50 - protein, 9 aa, Artificial Sequence
  Synthetic Construct
QQYNSFPPT

SEQ ID NO: 51 - protein, 445 aa, Artificial Sequence
  Synthetic Construct
QVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - protein, 214 aa, Artificial Sequence
  Synthetic Construct
DIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC